SEQUENCE LISTING (39 sequences)

SEQ ID NO: 1 - protein, 1265 aa, Severe acute respiratory syndrome coronavirus 2
MFVFLVLLPLVSSQCVNLTTRTQLPPAYTNSFTRGVYYPDKVFRSSVLHSTQDLFLPFFSNVTWFRAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKGGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 2 - protein, 122 aa, Artificial Sequence
  Description of Artificial Sequence Synthetic polypeptide
SQITLKESGPTLVKPTQTLTLTCTFSGFSLSTSGVGVGWIRQPPGKALEWLALIYWDDDKRYSPSLKSRLTITKDTSKNQVVLTMTNMDPVDTATYYCAHHKIERIFDYWGQGTLVTVSSAS

SEQ ID NO: 3 - protein, 109 aa, Artificial Sequence
  Description of Artificial Sequence Synthetic polypeptide
QSALAQPASVSGSPGQSITISCTGTSSDVGAYNYVSWYQQHPGKAPKLMIYDVSKRPSGVSNRFSGSKSGNTASLTISGLQAEDEADYYCSSYTTSSTVFGGGTKLTVL

SEQ ID NO: 4 - protein, 121 aa, Artificial Sequence
  Description of Artificial Sequence Synthetic polypeptide
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCTRAGWELRLDAFDIWGQGTMVTVSS

SEQ ID NO: 5 - protein, 106 aa, Artificial Sequence
  Description of Artificial Sequence Synthetic polypeptide
SSELTQDPAVSVALGQTVRITCQGDSLRSSYASWYQQKPGQAPILVIYDKNNRPSGIPDRFSGSSSGNTASLTITGAQAEDEADYYCNSRDSSGILFGGGTKLTVL

SEQ ID NO: 6 - protein, 128 aa, Artificial Sequence
  Description of Artificial Sequence Synthetic polypeptide
QVQLQESGPGLVKPSETLSLTCTVSGGSVSSSNYYWSWIRQPPGKGLEWIGYMYYSGSTKYNPSLKSRVTISVDTSKNQFSLKLSSVTAADTAVYYCAREVYYYDRSGYYASDGFDIWGQGTMVTVSS

SEQ ID NO: 7 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPQTFGQGTKVEIK

SEQ ID NO: 8 - protein, 117 aa, Artificial Sequence
  Description of Artificial Sequence Synthetic polypeptide
EVQLVESGGGLIQPGGSLRLSCAASGLTVSSNYMSWVRQAPGKGLEWVSVIYSGGSTFYADSVKGRFTISRDNSKNTLYLQMNSLRAEDTAVYYCARDLFYYGMDVWGQGTTVTVSS

SEQ ID NO: 9 - protein, 106 aa, Artificial Sequence
  Description of Artificial Sequence Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYPCFGPGTKVDIK

SEQ ID NO: 10 - protein, 129 aa, Artificial Sequence
  Description of Artificial Sequence Synthetic polypeptide
QVQLVQSGAEVKKPGASVRVSCKASGYTFTGYYMHWVRQAPGQGLEWMGWINPISDGTNYAQKFQGWVTMTRDTSISTVYMELSRLRSDDTAVYYCARGGSRCSGGNCYGWAYDAFDIWGQGTMITVSS

SEQ ID NO: 11 - protein, 110 aa, Artificial Sequence
  Description of Artificial Sequence Synthetic polypeptide
QSALTQPASVSGSPGQSITISCTGTSSDVGGYNFVSWYQQHPGKAPKLMIYDVSKRPSGVSNRFSGSKSGNTASLTISGLQAEDEADCYCSSYTSSSTFVFGTGTKVTVL

SEQ ID NO: 12 - protein, 129 aa, Artificial Sequence
  Description of Artificial Sequence Synthetic polypeptide
QVQLVQSGAEVKKPGASVKVSCKASGYTFTGYYMHWVRQAPGQGLEWMGWINPNSGGTNYAQKFQGRVTMTRDTSITTAYMELRRLRSDDTAVYYCARGLGVGCSGGNCYLDYYYMDVWGKGTTVTVSS

SEQ ID NO: 13 - protein, 110 aa, Artificial Sequence
  Description of Artificial Sequence Synthetic polypeptide
QSALTQPASVSGSPGQSITISCTGTSSDVGGYNYVSWYQQHPGKAPKLMIYDVSKRPSGVSNRFSGSKSGNTASLTISGLQAEDEGDYYCSSYTSSSTWVFGGGTKLTVL

SEQ ID NO: 14 - protein, 130 aa, Artificial Sequence
  Description of Artificial Sequence Synthetic polypeptide
EVHLVESGGGLVQPGGSLRLSCAASGFTFSDHYMDWVRQAPGKGLEWVGRTRNKANSYTTEYAASVKGRFTISRDDAKNSLYLQMNSLKTEDTAVYYCARVHRWAYCINGVCFGAYSDYWGQGTLVTVSS

SEQ ID NO: 15 - protein, 108 aa, Artificial Sequence
  Description of Artificial Sequence Synthetic polypeptide
EIVLTQSPGTLSLSPGERATLSCRASQSVSSSYLAWYQQKPGQAPRLLIYGASSRATGIPDRFSGSGSGTDFTLTISRLEPEDFAVYYCQQYGSSPSTFGQGTKLEIK

SEQ ID NO: 16 - protein, 119 aa, Artificial Sequence
  Description of Artificial Sequence Synthetic polypeptide
QVQLVESGGGVVQPGRSLRLSCAASGFTFSSYTMHWVRQAPGKGLEWVAAISYDGNNKYYADSVKGRFTISRDNSKNTLYLQMNSLRAEDTAVYYCARSILYGGGMDVWGQGTTVTVSS

SEQ ID NO: 17 - protein, 107 aa, Artificial Sequence
  Description of Artificial Sequence Synthetic polypeptide
DIQLTQSPSSLSASVGDRVTITCRASQGISSYLAWYQQKPGQAPKLLIYAASTLQSGVPSRFSGSGSGTDFTLTISSLQPEDFATYYCQQLNSYPLTFGGGTKVEIK

SEQ ID NO: 18 - protein, 125 aa, Artificial Sequence
  Description of Artificial Sequence Synthetic polypeptide
QVQLVQSGAEVKKAGSSVKVSCKASGGTFSSHTITWVRQAPGQGLEWMGRIIPILGIANYAQKFQGRVTITADKSTSTAYMELSSLRSEDTAVYYCASLQTVDTAIEKYYGMDVWGQGTTVTVSS

SEQ ID NO: 19 - protein, 106 aa, Artificial Sequence
  Description of Artificial Sequence Synthetic polypeptide
SYELTQPPSVSVSPGQTASITCSGDKLGDKYACWYQQKPGQSPVLVIYQDNKRPSGIPERFSGSNSGNTATLTISGTQAMDEADYYCQAWDSSTAVFGGGTKLTVL

SEQ ID NO: 20 - protein, 129 aa, Artificial Sequence
  Description of Artificial Sequence Synthetic polypeptide
QVQLVESGGGVVQPGRSLRLSCAASGLTFSSYVMHWVRQAPGKGLDWVGVIWYDGSKKYYADSVKGRFTISRDNSKNTLYLQMNSLRAEDTAVYYCARDPRDYYDFWSGYDYYYGLDVWGQGTTVTVSS

SEQ ID NO: 21 - protein, 109 aa, Artificial Sequence
  Description of Artificial Sequence Synthetic polypeptide
EIVLTQSPGILSLSPGERATLSCRASQSVSSSYLAWYQQKPGQAPRLLIYGASSRATGIPDRFSGSGSGTDFTLTISRLEPEDFAVYYCQQYGSSGALTFGGGTKVEIK

SEQ ID NO: 22 - protein, 128 aa, Artificial Sequence
  Description of Artificial Sequence Synthetic polypeptide
QVQLVQSGAEVKKPGASVKVSCKVSGYTLIELSMHWVRQAPGKGLEWMGGFDPEDAETIYAQKFQGRVTMTEDTSTDTAYMELSSLRSEDTAVYYCATGWAVAGSSDVWYYYYGMDVWGQGTTVTVSS

SEQ ID NO: 23 - protein, 110 aa, Artificial Sequence
  Description of Artificial Sequence Synthetic polypeptide
QSALTQPPSVSGSPGQSVTISCTGTSSDVGSYNRVSWYQQPPGTAPKLMIYEVSNRPSGVPDRFSGSKSGNTASLTISGLQAEDEADYYCSSYTSSSTYVFGTGTKVTVL

SEQ ID NO: 24 - protein, 128 aa, Artificial Sequence
  Description of Artificial Sequence Synthetic polypeptide
QVQLVQSGAEVKKPGASVKVSCKVSGYTLIELSMHWVRQAPGKGLEWMGGFDPEDAETIYAQKFQGRVTMTEDTSTDTAYMELSSLRSEDTAVYYCATGIAVIGPPPSTYYYYGMDVWGQGTTVTVSS

SEQ ID NO: 25 - protein, 110 aa, Artificial Sequence
  Description of Artificial Sequence Synthetic polypeptide
QSALTQPASVSGSPGQSITISCTGTSSDVGGYNYVSWYQQHPGKAPKLMIYDVSKRPSGVSNRFSGSKSGNTASLTISGLQAEDEADYYCSSYTSSSTYVFGTGTKVTVL

SEQ ID NO: 26 - protein, 121 aa, Artificial Sequence
  Description of Artificial Sequence Synthetic polypeptide
QVQLVQSGAEVKKPGASVKVSCKVSGYTLIELSMHWVRQAPGKGLEWMGGFDPEDVETIYAQQFQGRVTMTEDTSTDTAYMELSSLRSEDTAVYYCATGWAYKSTWYFGYWGQGTLVTVSS

SEQ ID NO: 27 - protein, 109 aa, Artificial Sequence
  Description of Artificial Sequence Synthetic polypeptide
QSALTQPPSASGSPGQSVTISCTGTSSDVGGYNYVSWYQQHPGKAPKLMIYEVSKRPSGVPDRFSGSKSGNTASLTVSGLQAEDEADYYCSSYAGSRMGFGGGTKLTVL

SEQ ID NO: 28 - protein, 124 aa, Artificial Sequence
  Description of Artificial Sequence Synthetic polypeptide
QVQLVQSGAEVKKPGSSVKVSCKASGGTFSSYAISWVRQAPGQGLEWMGGNIPIFGTANYAQKFQGRVTITADESTSTAYMELSSLRSEDTAVYYCARGVGYRGVIPLNWFDPWGQGTVVTVSS

SEQ ID NO: 29 - protein, 108 aa, Artificial Sequence
  Description of Artificial Sequence Synthetic polypeptide
EIVMTQSPATLSVSPGERATLSCRASQSVSSDLAWYQHKPGQAPRLLIYGASTRATGIPVRFSGSGSGTEFTLTISSLQSEDFAVYYCQQYNNWPPFTFGGGTKVEIK

SEQ ID NO: 30 - protein, 122 aa, Artificial Sequence
  Description of Artificial Sequence Synthetic polypeptide
QVQLVESGGGVVQPGRSLRLSCAASGFTFSSYGMHWVRQAPGKGLEWVAVISYDGSNKHYADSVKGRFTISRDNSKNTLYLQMNSLRAEDTAVYYCAKDSGYNYGYSWFDPWGQGTLVTVSS

SEQ ID NO: 31 - protein, 110 aa, Artificial Sequence
  Description of Artificial Sequence Synthetic polypeptide
SYELTQPPSVSVSPGQTARITCSADALAKQYAYWYQQKPGQAPVLVIYKDSERPSGIPERFSGSSSGTTVTLTISGVQAEDEADYYCQSTDNSGTYPNWVFGGGTKLTVL

SEQ ID NO: 32 - protein, 126 aa, Artificial Sequence
  Description of Artificial Sequence Synthetic polypeptide
QVQLQESGPGLVKPSETLSLTCTVSGGSISNYYWSWIRQSPGKGLEWIGYIYYSGSTNYNPSLKSRVTISVDTSKNQFSLKLSSVTAADTAVYYCARSAKHWLAPPGDYYYYMDVWGKGTTVTVSS

SEQ ID NO: 33 - protein, 106 aa, Artificial Sequence
  Description of Artificial Sequence Synthetic polypeptide
DIQLTQSPSFLSASVGDRVTITCRASQGISSYLAWYQQKPGKAPKLLIYAASTLQSGVPSRFSGSGSGTEFTLTISSLQPEDFATYYCQQLNSYLTFGGGTKVEIK

SEQ ID NO: 34 - protein, 134 aa, Artificial Sequence
  Description of Artificial Sequence Synthetic polypeptide
QVQLVQSGAEVKKPGASVKVSCKASGYTFTSYYMHWVRQAPGQGLEWMGVINPSGGSTSYAEKFRGRVTMTRDTSTSTVYMELSSLRSEDTAVYYCARDREPHSDSSGYWDSLKYYYYYALDVWGQGTTVTVSS

SEQ ID NO: 35 - protein, 107 aa, Artificial Sequence
  Description of Artificial Sequence Synthetic polypeptide
AIQLTQSPSSLSASVGDRVTITCRASQGISSYLAWYQQKPGKAPELLIYAASTLQSGVPSRFSGSGSGTDFTLTISSLQPEDFATYYCQQLNTYPFTFGPGTKVDIK

SEQ ID NO: 36 - protein, 21 aa, Artificial Sequence
  Description of Artificial Sequence Synthetic peptide
  MOD_RES (8)..(12): Any amino acid
ICASYQTXXXXXRRARSVASQ

SEQ ID NO: 37 - protein, 21 aa, Artificial Sequence
  Description of Artificial Sequence Synthetic peptide
ICASYQTQTNSPRRARSVASQ

SEQ ID NO: 38 - protein, 16 aa, Artificial Sequence
  Description of Artificial Sequence Synthetic peptide
ICASYQTRRARSVASQ

SEQ ID NO: 39 - protein, 9 aa, Artificial Sequence
  Description of Artificial Sequence Synthetic peptide
QTNSPRRAR

(19) United States
(12) Patent Application Publication
(10) Pub. No.: US 2024/0228594 A1
(43) Pub. Date: Jul. 11, 2024

(54) ANTIBODIES FOR THE TREATMENT AND PREVENTION OF COVID-19 AND EMERGING VARIANTS

(71) Applicant: The Trustees of Columbia University in the City of New York, New York, NY (US)

(72) Inventors: David D. HO, Bedford, NY (US); Yaoxing HUANG, Brooklyn, NY (US); Manoj S. NAIR, Fair Lawn, NJ (US)

(21) Appl. No.: 18/557,878

(22) PCT Filed: Apr. 27, 2022
(86) PCT No.: PCT/US2022/026516

§ 371 Date: Oct. 27, 2023

Related U.S. Application Data
(60) Provisional application No. 63/181,138, filed on Apr. 28, 2021.

(51) Int. Cl. C07K 16/10; A61K 39/00; A61K 39/215; A61P 31/14; A61P 37/04; C07K 14/005

(52) U.S. Cl. CPC C07K 16/1003; A61K 39/215; A61P 31/14; A61P 37/04; C07K 14/005; A61K 2039/53; A61K 2039/545

(57) ABSTRACT

The subject matter disclosed herein relates to a recombinant spike (S) protein from the B.1.351.2-7 variant of the SARS-CoV-2 virus, the DNA and RNA nucleotide sequences encoding the recombinant B.1.351.2-7 spike protein, and engineered antibodies that bind the B.1.351.2-7 spike protein and neutralize the B.1.351.2-7 virus.

This application claims the benefit of and priority to U.S. Provisional Patent Application No. 63/181,138, filed on Apr. 28, 2021, the content of which is hereby incorporated by reference in its entirety.

All patents, patent applications and publications cited herein are hereby incorporated by reference in their entirety. The disclosures of these publications in their entireties are hereby incorporated by reference into this application.

This patent disclosure contains material that is subject to copyright protection. The copyright owner has no objection to the facsimile reproduction by anyone of the patent document or the patent disclosure as it appears in the U.S. Patent and Trademark Office patent file or records, but otherwise reserves any and all copyright rights.

### BACKGROUND OF THE INVENTION

Coronavirus disease 2019 (COVID-19) is an infectious disease caused by the severe acute respiratory syndrome coronavirus 2 (SARS-COV-2). The disease affects multiple organs in the body including the lungs. There is an urgent need for the development of therapeutics to treat and prevent COVID-19.

### SUMMARY OF THE INVENTION

In certain aspects, the subject matter described herein provides an engineered antibody or a functional fragment thereof, wherein the antibody or fragment thereof selectively binds at least a portion of a SARS-COV-2 spike protein.

In some embodiments, the antibody or fragment comprises a first amino acid sequence having 50%, 55%, 60%, 65%, 70%, 75%, 80%, 85%, 90%, or 95% identity with SEQ ID NO: 4 and a second amino acid sequence having 50%, 55%, 60%, 65%, 70%, 75%, 80%, 85%, 90%, or 95% identity with SEQ ID NO: 5. In some embodiments, the antibody or fragment comprises a first amino acid sequence having 50%, 55%, 60%, 65%, 70%, 75%, 80%, 85%, 90%, or 95% identity with SEQ ID NO: 6 and a second amino acid sequence having 50%, 55%, 60%, 65%, 70%, 75%, 80%, 85%, 90%, or 95% identity with SEQ ID NO: 7. In some embodiments, the antibody or fragment comprises a first amino acid sequence having 50%, 55%, 60%, 65%, 70%, 75%, 80%, 85%, 90%, or 95% identity with SEQ ID NO: 8 and a second amino acid sequence having 50%, 55%, 60%, 65%, 70%, 75%, 80%, 85%, 90%, or 95% identity with SEQ ID NO: 9. In some embodiments, the antibody or fragment comprises a first amino acid sequence having 50%, 55%, 60%, 65%, 70%, 75%, 80%, 85%, 90%, or 95% identity with SEQ ID NO: 12 and a second amino acid sequence having 50%, 55%, 60%, 65%, 70%, 75%, 80%, 85%, 90%, or 95% identity with SEQ ID NO: 13. In some embodiments, the antibody or fragment comprises a first amino acid sequence having 50%, 55%, 60%, 65%, 70%, 75%, 80%, 85%, 90%, or 95% identity with SEQ ID NO: 16 and a second amino acid sequence having 50%, 55%, 60%, 65%, 70%, 75%, 80%, 85%, 90%, or 95% identity with SEQ ID NO: 17. In some embodiments, the antibody or fragment comprises a first amino acid sequence having 50%, 55%, 60%, 65%, 70%, 75%, 80%, 85%, 90%, or 95% identity with SEQ ID NO: 18 and a second amino acid sequence having 50%, 55%, 60%, 65%, 70%, 75%, 80%, 85%, 90%, or 95% identity with SEQ ID NO: 19. In some embodiments, the antibody or fragment comprises a first amino acid sequence having 50%, 55%, 60%, 65%, 70%, 75%, 80%, 85%, 90%, or 95% identity with SEQ ID NO: 20 and a second amino acid sequence having 50%, 55%, 60%, 65%, 70%, 75%, 80%, 85%, 90%, or 95% identity with SEQ ID NO: 21. In some embodiments, the antibody or fragment comprises a first amino acid sequence having 50%, 55%, 60%, 65%, 70%, 75%, 80%, 85%, 90%, or 95% identity with SEQ ID NO: 22 and a second amino acid sequence having 50%, 55%, 60%, 65%, 70%, 75%, 80%, 85%, 90%, or 95% identity with SEQ ID NO: 23. In some embodiments, the antibody or fragment comprises a first amino acid sequence having 50%, 55%, 60%, 65%, 70%, 75%, 80%, 85%, 90%, or 95% identity with SEQ ID NO: 24 and a second amino acid sequence having 50%, 55%, 60%, 65%, 70%, 75%, 80%, 85%, 90%, or 95% identity with SEQ ID NO: 25. In some embodiments, the antibody or fragment comprises a first amino acid sequence having 50%, 55%, 60%, 65%, 70%, 75%, 80%, 85%, 90%, or 95% identity with SEQ ID NO: 26 and a second amino acid sequence having 50%, 55%, 60%, 65%, 70%, 75%, 80%, 85%, 90%, or 95% identity with SEQ ID NO: 27. In some embodiments, the antibody or fragment comprises a first amino acid sequence having 50%, 55%, 60%, 65%, 70%, 75%, 80%, 85%, 90%, or 95% identity with SEQ ID NO: 32 and a second amino acid sequence having 50%, 55%, 60%, 65%, 70%, 75%, 80%, 85%, 90%, or 95% identity with SEQ ID NO: 33.

In some embodiments, the antibody or fragment comprises a first amino acid sequence having 50%, 55%, 60%, 65%, 70%, 75%, 80%, 85%, 90%, or 95% identity with SEQ ID NO: 2 and a second amino acid sequence having 50%, 55%, 60%, 65%, 70%, 75%, 80%, 85%, 90%, or 95% identity with SEQ ID NO: 3. In some embodiments, the antibody or fragment comprises a first amino acid sequence having 50%, 55%, 60%, 65%, 70%, 75%, 80%, 85%, 90%, or 95% identity with SEQ ID NO: 14 and a second amino acid sequence having 50%, 55%, 60%, 65%, 70%, 75%, 80%, 85%, 90%, or 95% identity with SEQ ID NO: 15. In some embodiments, the antibody or fragment comprises a first amino acid sequence having 50%, 55%, 60%, 65%, 70%, 75%, 80%, 85%, 90%, or 95% identity with SEQ ID NO: 10 and a second amino acid sequence having 50%, 55%, 60%, 65%, 70%, 75%, 80%, 85%, 90%, or 95% identity with SEQ ID NO: 11. In some embodiments, the antibody or fragment comprises a first amino acid sequence having 50%, 55%, 60%, 65%, 70%, 75%, 80%, 85%, 90%, or 95% identity with SEQ ID NO: 28 and a second amino acid sequence having 50%, 55%, 60%, 65%, 70%, 75%, 80%, 85%, 90%, or 95% identity with SEQ ID NO: 29. In some embodiments, the antibody or fragment comprises a first amino acid sequence having 50%, 55%, 60%, 65%, 70%, 75%, 80%, 85%, 90%, or 95% identity with SEQ ID NO: 30 and a second amino acid sequence having 50%, 55%, 60%, 65%, 70%, 75%, 80%, 85%, 90%, or 95% identity with SEQ ID NO: 31. In some embodiments, the antibody or fragment comprises a first amino acid sequence having 50%, 55%, 60%, 65%, 70%, 75%, 80%, 85%, 90%, or 95% identity with SEQ ID NO: 34 and a second amino acid sequence having 50%, 55%, 60%, 65%, 70%, 75%, 80%, 85%, 90%, or 95% identity with SEQ ID NO: 35.

In some embodiments, the antibody or fragment comprises a first amino acid sequence comprising SEQ ID NO: 4 and a second amino acid sequence comprising SEQ ID NO: 5. In some embodiments, the antibody or fragment comprises a first amino acid sequence comprising SEQ ID NO: 6 and a second amino acid sequence comprising SEQ ID NO: 7. In some embodiments, the antibody or fragment comprises a first amino acid sequence comprising SEQ ID NO: 8 and a second amino acid sequence comprising SEQ ID NO: 9. In some embodiments, the antibody or fragment comprises a first amino acid sequence comprising SEQ ID NO: 12 and a second amino acid sequence comprising SEQ ID NO: 13. In some embodiments, the antibody or fragment comprises a first amino acid sequence comprising SEQ ID NO: 16 and a second amino acid sequence comprising SEQ ID NO: 17. In some embodiments, the antibody or fragment comprises a first amino acid sequence comprising SEQ ID NO: 18 and a second amino acid sequence comprising SEQ ID NO: 19. In some embodiments, the antibody or fragment comprises a first amino acid sequence comprising SEQ ID NO: 20 and a second amino acid sequence comprising SEQ ID NO: 21. In some embodiments, the antibody or fragment comprises a first amino acid sequence comprising SEQ ID NO: 22 and a second amino acid sequence comprising SEQ ID NO: 23. In some embodiments, the antibody or fragment comprises a first amino acid sequence comprising SEQ ID NO: 24 and a second amino acid sequence comprising SEQ ID NO: 25. In some embodiments, the antibody or fragment comprises a first amino acid sequence comprising SEQ ID NO: 26 and a second amino acid sequence comprising SEQ ID NO: 27. In some embodiments, the antibody or fragment comprises a first amino acid sequence comprising SEQ ID NO: 32 and a second amino acid sequence comprising SEQ ID NO: 33.

In some embodiments, the antibody or fragment comprises a first amino acid sequence comprising SEQ ID NO: 2 and a second amino acid sequence comprising SEQ ID NO: 3. In some embodiments, the antibody or fragment comprises a first amino acid sequence comprising SEQ ID NO: 14 and a second amino acid sequence comprising SEQ ID NO: 15. In some embodiments, the antibody or fragment comprises a first amino acid sequence comprising SEQ ID NO: 10 and a second amino acid sequence comprising SEQ ID NO: 11. In some embodiments, the antibody or fragment comprises a first amino acid sequence comprising SEQ ID NO: 28 and a second amino acid sequence comprising SEQ ID NO: 29. In some embodiments, the antibody or fragment comprises a first amino acid sequence comprising SEQ ID NO: 30 and a second amino acid sequence comprising SEQ ID NO: 31. In some embodiments, the antibody or fragment comprises a first amino acid sequence comprising SEQ ID NO: 34 and a second amino acid sequence comprising SEQ ID NO: 35.

In some embodiments, the antibody or fragment comprises the complementarity-determining regions (CDRs) of SEQ ID NO: 4 and the complementarity-determining regions (CDRs) of SEQ ID NO: 5. In some embodiments, the antibody or fragment comprises the complementarity-determining regions (CDRs) of SEQ ID NO: 6 and the complementarity-determining regions (CDRs) of SEQ ID NO: 7. In some embodiments, the antibody or fragment comprises the complementarity-determining regions (CDRs) of SEQ ID NO: 8 and the complementarity-determining regions (CDRs) of SEQ ID NO: 9. In some embodiments, the antibody or fragment comprises the complementarity-determining regions (CDRs) of SEQ ID NO: 12 and the complementarity-determining regions (CDRs) of SEQ ID NO: 13. In some embodiments, the antibody or fragment comprises the complementarity-determining regions (CDRs) of SEQ ID NO: 16 and the complementarity-determining regions (CDRs) of SEQ ID NO: 17. In some embodiments, the antibody or fragment comprises the complementarity-determining regions (CDRs) of SEQ ID NO: 18 and the complementarity-determining regions (CDRs) of SEQ ID NO: 19. In some embodiments, the antibody or fragment comprises the complementarity-determining regions (CDRs) of SEQ ID NO: 20 and the complementarity-determining regions (CDRs) of SEQ ID NO: 21. In some embodiments, the antibody or fragment comprises the complementarity-determining regions (CDRs) of SEQ ID NO: 22 and the complementarity-determining regions (CDRs) of SEQ ID NO: 23. In some embodiments, the antibody or fragment comprises the complementarity-determining regions (CDRs) of SEQ ID NO: 24 and the complementarity-determining regions (CDRs) of SEQ ID NO: 25. In some embodiments, the antibody or fragment comprises the complementarity-determining regions (CDRs) of SEQ ID NO: 26 and the complementarity-determining regions (CDRs) of SEQ ID NO: 27. In some embodiments, the antibody or fragment comprises the complementarity-determining regions (CDRs) of SEQ ID NO: 32 and the complementarity-determining regions (CDRs) of SEQ ID NO: 33.

In some embodiments, the antibody or fragment comprises the complementarity-determining regions (CDRs) of SEQ ID NO: 2 and the complementarity-determining regions (CDRs) of SEQ ID NO: 3. In some embodiments, the antibody or fragment comprises the complementarity-determining regions (CDRs) of SEQ ID NO: 14 and the complementarity-determining regions (CDRs) of SEQ ID NO: 15. In some embodiments, the antibody or fragment comprises the complementarity-determining regions (CDRs) of SEQ ID NO: 10 and the complementarity-determining regions (CDRs) of SEQ ID NO: 11. In some embodiments, the antibody or fragment comprises the complementarity-determining regions (CDRs) of SEQ ID NO: 28 and the complementarity-determining regions (CDRs) of SEQ ID NO: 29. In some embodiments, the antibody or fragment comprises the complementarity-determining regions (CDRs) of SEQ ID NO: 30 and the complementarity-determining regions (CDRs) of SEQ ID NO: 31. In some embodiments, the antibody or fragment comprises the complementarity-determining regions (CDRs) of SEQ ID NO: 34 and the complementarity-determining regions (CDRs) of SEQ ID NO: 35.

In some embodiments, the SARS-COV-2 spike protein is a wild-type SARS-COV-2 WA1 spike protein. In some embodiments, the SARS-COV-2 spike protein is a SARS-COV-2 B.1.351 variant spike protein. In some embodiments, the SARS-COV-2 spike protein is a SARS-COV-2 B.1.351.2-7 variant spike protein. In some embodiments, the SARS-COV-2

In some embodiments, the antibody neutralizes a SARS-COV-2 virus. In some embodiments, the antibody neutralizes a SARS-COV-2 B.1.351 variant virus. In some embodiments, the antibody a SARS-COV-2 B.1.351.2-7 variant virus.

In certain aspects, the subject matter described herein provides a method for treating a subject having a viral infection or preventing a subject from developing a viral infection, the method comprising administering to the subject a therapeutically effective amount of a composition comprising any engineered monoclonal antibody described herein, wherein the antibody or fragment selectively binds at least a portion of a SARS-COV-2 spike protein.

In some embodiments, the SARS-COV-2 spike protein is a wild-type SARS-COV-2 WA1 spike protein. In some embodiments, the SARS-COV-2 spike protein is a SARS-COV-2 B.1.351 variant spike protein. In some embodiments, the SARS-COV-2 spike protein is a SARS-COV-2 B.1.351.2-7 variant spike protein. In some embodiments, the SARS-COV-2 spike protein is a SARS-COV-2 B.1.351.2-7 variant spike protein comprising SEQ ID NO: 1. In some embodiments, the antibody neutralizes a SARS-COV-2 virus. In some embodiments, the antibody neutralizes a SARS-COV-2 B.1.351 variant virus. In some embodiments, the antibody a SARS-COV-2 B.1.351.2-7 variant virus.

In certain aspects, the subject matter described herein provides a purified protein, wherein the protein comprises at least a portion of a SARS-COV-2 surface protein.

In some embodiments, the at least a portion of a SARS-COV-2 surface protein is a B.1.351.2-7 variant surface protein. In some embodiments, the B.1.351.2-7 variant surface protein is a spike protein (S). In some embodiments, the spike protein comprises an amino acid sequence having 50%, 55%, 60%, 65%, 70%, 75%, 80%, 85%, 90%, or 95% identity with SEQ ID NO: 1. In some embodiments, the spike protein comprises SEQ ID NO: 1.

In certain aspects, the subject matter described herein provides a synthetic nucleic acid sequence comprising a nucleotide sequence encoding a SARS-COV-2 B.1.351.2-7 variant spike protein.

In some embodiments, the nucleic acid sequence is a complementary deoxyribonucleic acid (cDNA) encoding for a SARS-COV-2 B.1.351.2-7 variant spike protein comprising SEQ ID NO: 1. In some embodiments, the nucleic acid sequence is a messenger ribonucleic acid (mRNA) encoding for a SARS-COV-2 B.1.351.2-7 variant spike protein comprising SEQ ID NO: 1. In some embodiments, the nucleic acid sequence encodes a B.1.351.2-7 variant spike protein comprising an amino acid sequence having 50%, 55%, 60%, 65%, 70%, 75%, 80%, 85%, 90%, or 95% identity with SEQ ID NO: 1. In some embodiments, the nucleic acid sequence encodes a B.1.351.2-7 variant spike protein comprising SEQ ID NO: 1.

In certain aspects, the subject matter described herein provides a pharmaceutical composition comprising a purified spike protein encoded by an amino acid sequence defined by SEQ ID NO: 1 or a portion thereof and a carrier, wherein the composition induces an immune response in a subject in need thereof.

In certain aspects, the subject matter described herein provides a pharmaceutical composition comprising: (i) a synthetic nucleic acid sequence comprising a nucleotide sequence encoding a SARS-COV-2 B.1.351.2-7 variant spike protein or a portion thereof; and (ii) a carrier.

In some embodiments, the nucleic acid sequence is a complementary deoxyribonucleic acid (cDNA) encoding for the SARS-COV-2 B.1.351.2-7 variant spike protein comprising SEQ ID NO: 1. In some embodiments, the nucleic acid sequence is a messenger ribonucleic acid (mRNA) encoding for the SARS-COV-2 B.1.351.2-7 variant spike protein comprising SEQ ID NO: 1. In some embodiments, the nucleic acid sequence encodes a B.1.351.2-7 variant spike protein comprising an amino acid sequence having 50%, 55%, 60%, 65%, 70%, 75%, 80%, 85%, 90%, or 95% identity with SEQ ID NO: 1. In some embodiments, the nucleic acid sequence encodes a B.1.351.2-7 variant spike protein comprising SEQ ID NO: 1. In some embodiments, the composition further comprises an adjuvant.

In certain aspects, the subject matter described herein provides a method of inducing an immune response in a subject in need thereof, the method comprising administering to the subject the any composition described herein. In some embodiments, the subject is at risk of contracting a SARS-COV-2 virus. In some embodiments, the SARS-COV-2 virus is a WA1 strain. In some embodiments, the SARS-COV-2 virus is a B.1.351 strain. In some embodiments the SARS-COV-2 virus is a B.1.351.2-7 strain. In some embodiments, the method comprises a prime-boost regimen of administering the composition. In some embodiments, the prime and the boost comprise the same composition. In some embodiments, the prime and the boost comprise a different composition.

### BRIEF DESCRIPTION OF FIGURES

The patent or application file contains at least one drawing originally in color. To conform to the requirements for PCT patent applications, many of the figures presented herein are black and white representations of images originally created in color.

FIGS. 1A-T show neutralization of mAb 2-7-resistant SARS-COV-2 B.1.351 virus strain (B.1.351.2-7) with monoclonal antibodies (mAb) to the receptor binding domain (RBD) of the B.1.351.2-7 spike protein (S). FIG. 1A shows neutralization for mAb 2-7. FIG. 1B shows neutralization for mAb REGN10987. FIG. 1C shows neutralization for mAb 2-38. FIG. 1D shows neutralization for mAb C135. FIG. 1E shows neutralization for mAb CoV2-2130. FIG. 1F shows neutralization for mAb 2-36. FIG. 1G shows neutralization for mAb COVA 1-16. FIG. 1H shows neutralization for mAb 1-20. FIG. 1I shows neutralization for mAb 2-15. FIG. 1J shows neutralization for mAb 2-43. FIG. 1K shows neutralization for mAb C121. FIG. 1L shows neutralization for mAb LY CoV 555. FIG. 1M shows neutralization for mAb 2-30. FIG. 1N shows neutralization for mAb REGN10985. FIG. 1O shows neutralization for mAb CB6. FIG. 1P shows neutralization for mAb 1-57. FIG. 1Q shows neutralization for mAb REGN10985. FIG. 1R shows neutralization for mAb S309. FIG. 1S shows neutralization for mAb CoV2-2196. FIG. 1T shows neutralization for mAb CR3022.

FIGS. 2A-J show neutralization of mAb 2-7-resistant SARS-COV-2 B.1.351 virus strain (B.1.351.2-7) with monoclonal antibodies to the N-terminal domain (NTD) of the B.1.351.2-7 spike protein. FIG. 2A shows neutralization for mAb 4A8. FIG. 2B shows neutralization for mAb 4-8. FIG. 2C shows neutralization for mAb 5-24. FIG. 2D shows neutralization for mAb 1-68. FIG. 2E shows neutralization for mAb 1-87. FIG. 2F shows neutralization for mAb 2-51. FIG. 2G shows neutralization for mAb 2-17. FIG. 2H shows neutralization for mAb 4-18. FIG. 2I shows neutralization for mAb 4-19. FIG. 2J shows neutralization for mAb 5-7.

FIG. 3 shows a range of neutralization potency (IC₅₀ μg/ml) of spike protein RBD-specific mAbs to the SARS-COV-2 WA1 (wild-type), B.1.351 (beta variant), and B.1.351.2-7 virus strains.

FIG. 4 shows neutralization potency (IC₅₀ μg/ml) of spike protein NTD-specific mAbs to the WA1, B.1.351, and the B. 1.351.2-7 virus strains.

FIG. 5 shows a schematic representation of B.1.351.2-7 variant strain mutations.

FIG. 6 shows the sequence of the B.1.351.2-7 spike protein. Yellow highlight indicates the NTD. Green font indicates the RBD. Dark blue font indicates the S1/S2 domains. Light blue font is the FP domain. Orange font indicates the HR1 domain. Brown font indicates the HR2 domain. Purple font is the TM domain. Pink font is the CT domain.

### DETAILED DESCRIPTION OF THE INVENTION

In certain aspects, the subject matter described herein provides an engineered antibody or a functional fragment thereof, wherein the antibody or fragment thereof selectively binds at least a portion of a SARS-COV-2 spike protein.

In some embodiments, the antibody or fragment comprises a first amino acid sequence having 50%, 55%, 60%, 65%, 70%, 75%, 80%, 85%, 90%, or 95% identity with SEQ ID NO: 4 and a second amino acid sequence having 50%, 55%, 60%, 65%, 70%, 75%, 80%, 85%, 90%, or 95% identity with SEQ ID NO: 5. In some embodiments, the antibody or fragment comprises a first amino acid sequence having 50%, 55%, 60%, 65%, 70%, 75%, 80%, 85%, 90%, or 95% identity with SEQ ID NO: 6 and a second amino acid sequence having 50%, 55%, 60%, 65%, 70%, 75%, 80%, 85%, 90%, or 95% identity with SEQ ID NO: 7. In some embodiments, the antibody or fragment comprises a first amino acid sequence having 50%, 55%, 60%, 65%, 70%, 75%, 80%, 85%, 90%, or 95% identity with SEQ ID NO: 8 and a second amino acid sequence having 50%, 55%, 60%, 65%, 70%, 75%, 80%, 85%, 90%, or 95% identity with SEQ ID NO: 9. In some embodiments, the antibody or fragment comprises a first amino acid sequence having 50%, 55%, 60%, 65%, 70%, 75%, 80%, 85%, 90%, or 95% identity with SEQ ID NO: 12 and a second amino acid sequence having 50%, 55%, 60%, 65%, 70%, 75%, 80%, 85%, 90%, or 95% identity with SEQ ID NO: 13. In some embodiments, the antibody or fragment comprises a first amino acid sequence having 50%, 55%, 60%, 65%, 70%, 75%, 80%, 85%, 90%, or 95% identity with SEQ ID NO: 16 and a second amino acid sequence having 50%, 55%, 60%, 65%, 70%, 75%, 80%, 85%, 90%, or 95% identity with SEQ ID NO: 17. In some embodiments, the antibody or fragment comprises a first amino acid sequence having 50%, 55%, 60%, 65%, 70%, 75%, 80%, 85%, 90%, or 95% identity with SEQ ID NO: 18 and a second amino acid sequence having 50%, 55%, 60%, 65%, 70%, 75%, 80%, 85%, 90%, or 95% identity with SEQ ID NO: 19. In some embodiments, the antibody or fragment comprises a first amino acid sequence having 50%, 55%, 60%, 65%, 70%, 75%, 80%, 85%, 90%, or 95% identity with SEQ ID NO: 20 and a second amino acid sequence having 50%, 55%, 60%, 65%, 70%, 75%, 80%, 85%, 90%, or 95% identity with SEQ ID NO: 21. In some embodiments, the antibody or fragment comprises a first amino acid sequence having 50%, 55%, 60%, 65%, 70%, 75%, 80%, 85%, 90%, or 95% identity with SEQ ID NO: 22 and a second amino acid sequence having 50%, 55%, 60%, 65%, 70%, 75%, 80%, 85%, 90%, or 95% identity with SEQ ID NO: 23. In some embodiments, the antibody or fragment comprises a first amino acid sequence having 50%, 55%, 60%, 65%, 70%, 75%, 80%, 85%, 90%, or 95% identity with SEQ ID NO: 24 and a second amino acid sequence having 50%, 55%, 60%, 65%, 70%, 75%, 80%, 85%, 90%, or 95% identity with SEQ ID NO: 25. In some embodiments, the antibody or fragment comprises a first amino acid sequence having 50%, 55%, 60%, 65%, 70%, 75%, 80%, 85%, 90%, or 95% identity with SEQ ID NO: 26 and a second amino acid sequence having 50%, 55%, 60%, 65%, 70%, 75%, 80%, 85%, 90%, or 95% identity with SEQ ID NO: 27. In some embodiments, the antibody or fragment comprises a first amino acid sequence having 50%, 55%, 60%, 65%, 70%, 75%, 80%, 85%, 90%, or 95% identity with SEQ ID NO: 32 and a second amino acid sequence having 50%, 55%, 60%, 65%, 70%, 75%, 80%, 85%, 90%, or 95% identity with SEQ ID NO: 33.

In some embodiments, the antibody or fragment comprises a first amino acid sequence having 50%, 55%, 60%, 65%, 70%, 75%, 80%, 85%, 90%, or 95% identity with SEQ ID NO: 2 and a second amino acid sequence having 50%, 55%, 60%, 65%, 70%, 75%, 80%, 85%, 90%, or 95% identity with SEQ ID NO: 3. In some embodiments, the antibody or fragment comprises a first amino acid sequence having 50%, 55%, 60%, 65%, 70%, 75%, 80%, 85%, 90%, or 95% identity with SEQ ID NO: 14 and a second amino acid sequence having 50%, 55%, 60%, 65%, 70%, 75%, 80%, 85%, 90%, or 95% identity with SEQ ID NO: 15. In some embodiments, the antibody or fragment comprises a first amino acid sequence having 50%, 55%, 60%, 65%, 70%, 75%, 80%, 85%, 90%, or 95% identity with SEQ ID NO: 10 and a second amino acid sequence having 50%, 55%, 60%, 65%, 70%, 75%, 80%, 85%, 90%, or 95% identity with SEQ ID NO: 11. In some embodiments, the antibody or fragment comprises a first amino acid sequence having 50%, 55%, 60%, 65%, 70%, 75%, 80%, 85%, 90%, or 95% identity with SEQ ID NO: 28 and a second amino acid sequence having 50%, 55%, 60%, 65%, 70%, 75%, 80%, 85%, 90%, or 95% identity with SEQ ID NO: 29. In some embodiments, the antibody or fragment comprises a first amino acid sequence having 50%, 55%, 60%, 65%, 70%, 75%, 80%, 85%, 90%, or 95% identity with SEQ ID NO: 30 and a second amino acid sequence having 50%, 55%, 60%, 65%, 70%, 75%, 80%, 85%, 90%, or 95% identity with SEQ ID NO: 31. In some embodiments, the antibody or fragment comprises a first amino acid sequence having 50%, 55%, 60%, 65%, 70%, 75%, 80%, 85%, 90%, or 95% identity with SEQ ID NO: 34 and a second amino acid sequence having 50%, 55%, 60%, 65%, 70%, 75%, 80%, 85%, 90%, or 95% identity with SEQ ID NO: 35.

In some embodiments, the antibody or fragment comprises a first amino acid sequence comprising SEQ ID NO: 4 and a second amino acid sequence comprising SEQ ID NO: 5. In some embodiments, the antibody or fragment comprises a first amino acid sequence comprising SEQ ID NO: 6 and a second amino acid sequence comprising SEQ ID NO: 7. In some embodiments, the antibody or fragment comprises a first amino acid sequence comprising SEQ ID NO: 8 and a second amino acid sequence comprising SEQ ID NO: 9. In some embodiments, the antibody or fragment comprises a first amino acid sequence comprising SEQ ID NO: 12 and a second amino acid sequence comprising SEQ ID NO: 13. In some embodiments, the antibody or fragment comprises a first amino acid sequence comprising SEQ ID NO: 16 and a second amino acid sequence comprising SEQ ID NO: 17. In some embodiments, the antibody or fragment comprises a first amino acid sequence comprising SEQ ID NO: 18 and a second amino acid sequence comprising SEQ ID NO: 19. In some embodiments, the antibody or fragment comprises a first amino acid sequence comprising SEQ ID NO: 20 and a second amino acid sequence comprising SEQ ID NO: 21. In some embodiments, the antibody or fragment comprises a first amino acid sequence comprising SEQ ID NO: 22 and a second amino acid sequence comprising SEQ ID NO: 23. In some embodiments, the antibody or fragment comprises a first amino acid sequence comprising SEQ ID NO: 24 and a second amino acid sequence comprising SEQ ID NO: 25. In some embodiments, the antibody or fragment comprises a first amino acid sequence comprising SEQ ID NO: 26 and a second amino acid sequence comprising SEQ ID NO: 27. In some embodiments, the antibody or fragment comprises a first amino acid sequence comprising SEQ ID NO: 32 and a second amino acid sequence comprising SEQ ID NO: 33.

In some embodiments, the antibody or fragment comprises a first amino acid sequence comprising SEQ ID NO: 2 and a second amino acid sequence comprising SEQ ID NO: 3. In some embodiments, the antibody or fragment comprises a first amino acid sequence comprising SEQ ID NO: 14 and a second amino acid sequence comprising SEQ ID NO: 15. In some embodiments, the antibody or fragment comprises a first amino acid sequence comprising SEQ ID NO: 10 and a second amino acid sequence comprising SEQ ID NO: 11. In some embodiments, the antibody or fragment comprises a first amino acid sequence comprising SEQ ID NO: 28 and a second amino acid sequence comprising SEQ ID NO: 29. In some embodiments, the antibody or fragment comprises a first amino acid sequence comprising SEQ ID NO: 30 and a second amino acid sequence comprising SEQ ID NO: 31. In some embodiments, the antibody or fragment comprises a first amino acid sequence comprising SEQ ID NO: 34 and a second amino acid sequence comprising SEQ ID NO: 35.

In some embodiments, the antibody or fragment comprises the complementarity-determining regions (CDRs) of SEQ ID NO: 4 and the complementarity-determining regions (CDRs) of SEQ ID NO: 5. In some embodiments, the antibody or fragment comprises the complementarity-determining regions (CDRs) of SEQ ID NO: 6 and the complementarity-determining regions (CDRs) of SEQ ID NO: 7. In some embodiments, the antibody or fragment comprises the complementarity-determining regions (CDRs) of SEQ ID NO: 8 and the complementarity-determining regions (CDRs) of SEQ ID NO: 9. In some embodiments, the antibody or fragment comprises the complementarity-determining regions (CDRs) of SEQ ID NO: 12 and the complementarity-determining regions (CDRs) of SEQ ID NO: 13. In some embodiments, the antibody or fragment comprises the complementarity-determining regions (CDRs) of SEQ ID NO: 16 and the complementarity-determining regions (CDRs) of SEQ ID NO: 17. In some embodiments, the antibody or fragment comprises the complementarity-determining regions (CDRs) of SEQ ID NO: 18 and the complementarity-determining regions (CDRs) of SEQ ID NO: 19. In some embodiments, the antibody or fragment comprises the complementarity-determining regions (CDRs) of SEQ ID NO: 20 and the complementarity-determining regions (CDRs) of SEQ ID NO: 21. In some embodiments, the antibody or fragment comprises the complementarity-determining regions (CDRs) of SEQ ID NO: 22 and the complementarity-determining regions (CDRs) of SEQ ID NO: 23. In some embodiments, the antibody or fragment comprises the complementarity-determining regions (CDRs) of SEQ ID NO: 24 and the complementarity-determining regions (CDRs) of SEQ ID NO: 25. In some embodiments, the antibody or fragment comprises the complementarity-determining regions (CDRs) of SEQ ID NO: 26 and the complementarity-determining regions (CDRs) of SEQ ID NO: 27. In some embodiments, the antibody or fragment comprises the complementarity-determining regions (CDRs) of SEQ ID NO: 32 and the complementarity-determining regions (CDRs) of SEQ ID NO: 33.

In some embodiments, the antibody or fragment comprises the complementarity-determining regions (CDRs) of SEQ ID NO: 2 and the complementarity-determining regions (CDRs) of SEQ ID NO: 3. In some embodiments, the antibody or fragment comprises the complementarity-determining regions (CDRs) of SEQ ID NO: 14 and the complementarity-determining regions (CDRs) of SEQ ID NO: 15. In some embodiments, the antibody or fragment comprises the complementarity-determining regions (CDRs) of SEQ ID NO: 10 and the complementarity-determining regions (CDRs) of SEQ ID NO: 11. In some embodiments, the antibody or fragment comprises the complementarity-determining regions (CDRs) of SEQ ID NO: 28 and the complementarity-determining regions (CDRs) of SEQ ID NO: 29. In some embodiments, the antibody or fragment comprises the complementarity-determining regions (CDRs) of SEQ ID NO: 30 and the complementarity-determining regions (CDRs) of SEQ ID NO: 31. In some embodiments, the antibody or fragment comprises the complementarity-determining regions (CDRs) of SEQ ID NO: 34 and the complementarity-determining regions (CDRs) of SEQ ID NO: 35.

In some embodiments, the SARS-COV-2 spike protein is a wild-type SARS-COV-2 WA1 spike protein. In some embodiments, the SARS-COV-2 spike protein is a SARS-COV-2 B.1.351 variant spike protein. In some embodiments, the SARS-COV-2 spike protein is a SARS-COV-2 B.1.351.2-7 variant spike protein. In some embodiments, the SARS-COV-2 spike protein is a SARS-COV-2 B.1.351.2-7 variant spike protein comprising SEQ ID NO: 1.

In some embodiments, the antibody neutralizes a SARS-COV-2 virus. In some embodiments, the antibody neutralizes a SARS-COV-2 B.1.351 variant virus. In some embodiments, the antibody a SARS-COV-2 B.1.351.2-7 variant virus.

In certain aspects, the subject matter described herein provides a method for treating a subject having a viral infection or preventing a subject from developing a viral infection, the method comprising administering to the subject a therapeutically effective amount of a composition comprising any engineered monoclonal antibody described herein, wherein the antibody or fragment selectively binds at least a portion of a SARS-COV-2 spike protein.

In some embodiments, the SARS-COV-2 spike protein is a wild-type SARS-COV-2 WA1 spike protein. In some embodiments, the SARS-COV-2 spike protein is a SARS-COV-2 B.1.351 variant spike protein. In some embodiments, the SARS-COV-2 spike protein is a SARS-COV-2 B.1.351.2-7 variant spike protein. In some embodiments, the SARS-COV-2 In some embodiments, the antibody neutralizes a SARS-COV-2 virus. In some embodiments, the antibody neutralizes a SARS-COV-2 B.1.351 variant virus. In some embodiments, the antibody a SARS-COV-2 B.1.351.2-7 variant virus.

In certain aspects, the subject matter described herein provides a purified protein, wherein the protein comprises at least a portion of a SARS-COV-2 surface protein.

In some embodiments, the at least a portion of a SARS-COV-2 surface protein is a B.1.351.2-7 variant surface protein. In some embodiments, the B.1.351.2-7 variant surface protein is a spike protein (S). In some embodiments, the spike protein comprises an amino acid sequence having 50%, 55%, 60%, 65%, 70%, 75%, 80%, 85%, 90%, or 95% identity with SEQ ID NO: 1. In some embodiments, the spike protein comprises SEQ ID NO: 1.

In certain aspects, the subject matter described herein provides a synthetic nucleic acid sequence comprising a nucleotide sequence encoding a SARS-COV-2 B.1.351.2-7 variant spike protein.

In some embodiments, the nucleic acid sequence is a complementary deoxyribonucleic acid (cDNA) encoding for a SARS-COV-2 B.1.351.2-7 variant spike protein comprising SEQ ID NO: 1. In some embodiments, the nucleic acid sequence is a messenger ribonucleic acid (mRNA) encoding for a SARS-COV-2 B.1.351.2-7 variant spike protein comprising SEQ ID NO: 1. In some embodiments, the nucleic acid sequence encodes a B.1.351.2-7 variant spike protein comprising an amino acid sequence having 50%, 55%, 60%, 65%, 70%, 75%, 80%, 85%, 90%, or 95% identity with SEQ ID NO: 1. In some embodiments, the nucleic acid sequence encodes a B.1.351.2-7 variant spike protein comprising SEQ ID NO: 1.

In certain aspects, the subject matter described herein provides a pharmaceutical composition comprising a purified spike protein encoded by an amino acid sequence defined by SEQ ID NO: 1 or a portion thereof and a carrier, wherein the composition induces an immune response in a subject in need thereof.

In certain aspects, the subject matter described herein provides a pharmaceutical composition comprising: (i) a synthetic nucleic acid sequence comprising a nucleotide sequence encoding a SARS-COV-2 B.1.351.2-7 variant spike protein or a portion thereof; and (ii) a carrier.

In some embodiments, the nucleic acid sequence is a complementary deoxyribonucleic acid (cDNA) encoding for the SARS-COV-2 B.1.351.2-7 variant spike protein comprising SEQ ID NO: 1. In some embodiments, the nucleic acid sequence is a messenger ribonucleic acid (mRNA) encoding for the SARS-COV-2 B.1.351.2-7 variant spike protein comprising SEQ ID NO: 1. In some embodiments, the nucleic acid sequence encodes a B.1.351.2-7 variant spike protein comprising an amino acid sequence having 50%, 55%, 60%, 65%, 70%, 75%, 80%, 85%, 90%, or 95% identity with SEQ ID NO: 1. In some embodiments, the nucleic acid sequence encodes a B.1.351.2-7 variant spike protein comprising SEQ ID NO: 1. In some embodiments, the composition further comprises an adjuvant.

In certain aspects, the subject matter described herein provides a method of inducing an immune response in a subject in need thereof, the method comprising administering to the subject the any composition described herein. In some embodiments, the subject is at risk of contracting a SARS-COV-2 virus. In some embodiments, the SARS-COV-2 virus is a WA1 strain. In some embodiments, the SARS-COV-2 virus is a B.1.351 strain. In some embodiments the SARS-COV-2 virus is a B.1.351.2-7 strain. In some embodiments, the method comprises a prime-boost regimen of administering the composition. In some embodiments, the prime and the boost comprise the same composition. In some embodiments, the prime and the boost comprise a different composition.

In some embodiments, the subject matter described herein related to monoclonal antibodies (mAb) specific for wild-type SAR-COV-2 (WA1) and variant strains including, but not limited to SARS-COV-2 B.1.351 or B.1.351.2-7. In some embodiments, the B. 1.351.2-7 virus strain is resistant to the neutralizing effects of the mAb 2-7. During the study of emerging COVID-19 variants, it was observed that the neutralization activities of many monoclonal antibodies, mAb therapeutics for FDA emergency use authorization (EUA), and many clinical development candidates, are significantly impacted by the SARS-COV-2 pan-resistant virus strain designated as B.1.351.2-7. This resistant virus strain was identified using the monoclonal antibody 2-7 in vitro selection. Antibody 2-7 is a receptor binding domain (RBD), neutralizing monoclonal antibody that shows potent neutralization activities against multiple SARS-COV-2 variants, including the B.1.351 strain.

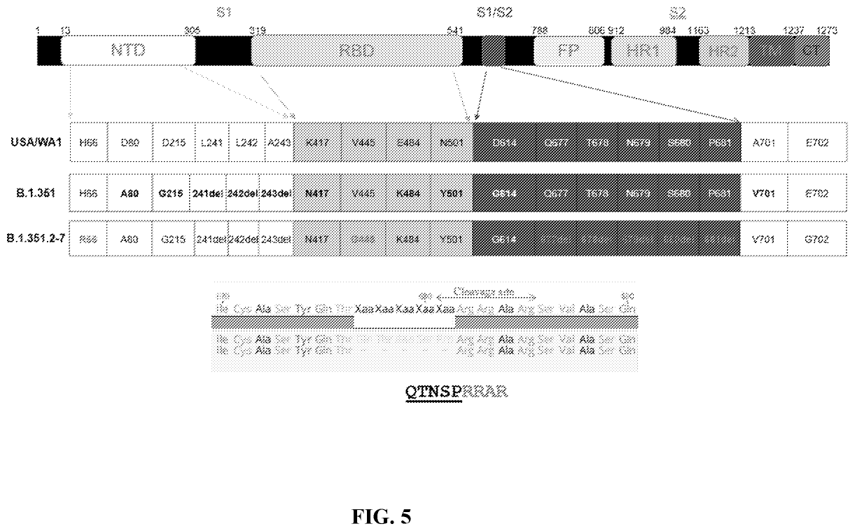

In some embodiments, the subject matter described herein relates to the isolation and sequencing of the resistant B.1.351.2-7 variant virus. The key mutations in its spike protein sequence include H66R, V445G, and a 677-681 deletion as shown in FIG. 5. A pseudovirus neutralization assay will be performed to dissect which point mutations, or a combination of the mutations, contribute to the resistant phenotype. In some embodiments, the subject matter described herein relates to B.1.351.2-7 variant virus neutralization assays using a panel of monoclonal antibodies. In some embodiments, the mAbs are specific for the RBD of the spike protein of the B.1.351.2-7 variant virus. In some embodiments, the mAbs are specific for the NTD of the spike protein of the B. 1.351.2-7 variant virus. Convalescent sera and vaccine sera will be utilized to assess the potential impact of this resistant virus on currently available therapeutics and vaccines.

As the B.1.351.2-7 virus is pan-resistant to most of the neutralizing antibodies and current EUA therapeutic antibodies, the effectiveness of current COVID-19 therapies and vaccines can be severely impacted by emerging variants sharing similar sequences as the pan-resistant viral strain disclosed herein. In one embodiment, the subject matter disclosed herein relates to development of vaccine candidates to inoculate subjects against the B.1.351.2-7 strain. In one embodiment, the subject matter disclosed herein relates to isolating antibody therapeutics that effective against the B.1.351.2-7 resistant strain. In one embodiment, the subject matter disclosed herein relates to the prevention and treatment of future COVID-19 outbreaks caused by resistant SARS-COV-2 variants. In one embodiment, the subject matter disclosed herein relates to designing vaccines against emerging SARS-COV-2 variants. In some embodiments, the subject matter disclosed herein relates to designing a bait protein for isolating neutralizing antibodies that show potent and/or cross-reactivity against the B.1.351.2-7 resistant strain and future pan-resistant variant of SARS-COV-2. In some embodiments, the bait protein is designed from at least a portion of a protein from the B.1.351.2-7 viral particle. In some embodiments, the subject matter disclosed herein relates to detection of viral variants.

Earlier passages will be sequenced and viral sequences that are pan-resistant to current neutralizing antibodies will be isolated. Key mutations contributing to the resistant phenotype will be identified by pseudovirus neutralization. Neutralization against additional panel of antibodies (both RBD and NTD), convalescent sera and vaccine sera will be performed to assess the potential impact of this resistant virus on therapeutics and vaccines. Spike trimer will be generated based on the resistant viral sequence for structural characterization and for immunization in a humanized mouse system or other animal models known in the art to generate the next generation of neutralizing antibodies as therapeutics.

SEQ ID NO: 1 is the amino acid sequence of variant B.1.351.2-7 full length spike protein:

MFVFLVLLPLVSSQCVNLTTRTQLPPAYTNSFTRGVYYPDKVFRSSVLHS
TQDLFLPFFSNVTWFRAIHVSGTNGTKRFANPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRGLPQGFSALEPLVDLPIGINITRFQTLHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAW
NRKRISNCVADYSVLYNSASFSTFKCYGVSPTKLNDLCFTNVYADSFVIR
GDEVRQIAPGQTGNIADYNYKLPDDFTGCVIAWNSNNLDSKGGGNYNYLY
RLFRKSNLKPFERDISTEIYQAGSTPCNGVKGFNCYFPLQSYGFQPTYGV
GYQPYRVVVLSFELLHAPATVCGPKKSTNLVKNKCVNFNFNGLTGTGVLT
ESNKKFLPFQQFGRDIADTTDAVRDPQTLEILDITPCSFGGVSVITPGTN
TSNQVAVLYQGVNCTEVPVAIHADQLTPTWRVYSTGSNVFQTRAGCLIGA
EHVNNSYECDIPIGAGICASYQTRRARSVASQSIIAYTMSLGVENSVAYS
NNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPD
PSKPSKRSFIEDLLFNKVTLADAGFIKQYGDCLGDIAARDLICAQKFNGL
TVLPPLLTDEMIAQYTSALLAGTITSGWTFGAGAALQIPFAMQMAYRENG
IGVTQNVLYENQKLIANQFNSAIGKIQDSLSSTASALGKLQDVVNQNAQA
LNTLVKQLSSNFGAISSVLNDILSRLDKVEAEVQIDRLITGRLQSLQTYV
TQQLIRAAEIRASANLAATKMSECVLGQSKRVDFCGKGYHLMSFPQSAPH
GVVFLHVTYVPAQEKNFTTAPAICHDGKAHFPREGVFVSNGTHWFVTQRN
FYEPQIITTDNTFVSGNCDVVIGIVNNTVYDPLQPELDSFKEELDKYFKN
HTSPDVDLGDISGINASVVNIQKEIDRLNEVAKNLNESLIDLQELGKYEQ
YIKWPWYIWLGFIAGLIAIVMVTIMLCCMTSCCSCLKGCCSCGSCCKFDE
DDSEPVLKGVKLHYT*

In some embodiments, the subject matter described herein relates to a purified variant B.1.351.2-7 spike protein. In some embodiment, a vector encoding a variant B.1.351.2-7 spike protein is introduced into a population of bacteria. In some embodiments, the protein is produced in a bacteria culture comprising the population of bacteria transform with the vector encoding a variant B.1.351.2-7 spike protein. In some embodiments, the protein is purified from the bacterial culture. In some embodiments, the vector comprises a vector backbone and a nucleic acid sequence encoding for an amino acid sequence having 50%, 55%, 60%, 65%, 70%, 75%, 80%, 85%, 90%, or 95% identity with SEQ ID NO:1. In some embodiments, the vector comprises a vector backbone and a nucleic acid sequence encoding for SEQ ID NO:1. In some embodiments, the vector backbone is any backbone used in the art for expression vectors. In some embodiments, the protein is purified using affinity chromatography. In some embodiments, the protein is purified using any suitable method known in the art. In some embodiments, the purified variant B.1.351.2-7 spike protein is a recombinant protein.

Sequences of mAbs to SARS-COV-2 B.1.351.2-7 Variant Spike Protein

Underlined and italicized amino acids in the following SEQ ID NOS represent the respective complementarity-determining regions (CDRs).

### Sequence of mAb 2-7

SEQ ID NO: 2 is the variable region of the heavy chain of antibody number 2-7, which specifically recognizes the RBD.

SQITLKESGPTLVKPTQTLTLTCTFSGFSLSTSGVGVGWIRQPPGKALEW
LALIYWDDDKRYSPSLKSRLTITKDTSKNQVVLTMTNMDPVDTATYYCAH
HKIERIFDYWGQGTLVTVSSAS

SEQ ID NO: 3 is the variable region of the light chain of antibody number 2-7, which specifically recognizes the RBD.

QSALAQPASVSGSPGQSITISCTGTSSDVGAYNYVSWYQQHPGKAPKLMI
YDVSKRPSGVSNRFSGSKSGNTASLTISGLQAEDEADYYCSSYTTSSTVF
GGGTKLTVL

### Sequence of mAb 2-38

SEQ ID NO: 4 is the variable region of the heavy chain of antibody number 2-38.

EVQLVESGGGLVKPGGSLRLSCAASGFTFSSYSMNWVRQAPGKGLEWVSS
ISSSSSYIYYADSVKGRFTISRDNAKNSLYLQMNSLRAEDTAVYYCTRAG
WELRLDAFDIWGQGTMVTVSS

SEQ ID NO: 5 is the variable region of the light chain of antibody number 2-38.

SSELTQDPAVSVALGQTVRITCQGDSLRSSYASWYQQKPGQAPILVIYDK
NNRPSGIPDRFSGSSSGNTASLTITGAQAEDEADYYCNSRDSSGILFGGG
TKLTVL

Sequence of mAb 2-36

SEQ ID NO: 6 is the variable region of the heavy chain of antibody number 2-36, which specifically recognizes the RBD.

QVQLQESGPGLVKPSETLSLTCTVSGGSVSSSNYYWSWIRQPPGKGLEWI
GYMYYSGSTKYNPSLKSRVTISVDTSKNQFSLKLSSVTAADTAVYYCARE
VYYYDRSGYYASDGFDIWGQGTMVTVSS

SEQ ID NO: 7 is the variable region of the light chain of antibody number 2-36, which specifically recognizes the RBD.

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPQTFG
QGTKVEIK

### Sequence of mAb 1-20

SEQ ID NO: 8 is the variable region of the heavy chain of antibody number 1-20, which specifically recognizes the RBD.

EVQLVESGGGLIQPGGSLRLSCAASGLTVSSNYMSWVRQAPGKGLEWVSV
IYSGGSTFYADSVKGRFTISRDNSKNTLYLQMNSLRAEDTAVYYCARDLF
YYGMDVWGQGTTVTVSS

SEQ ID NO: 9 is the variable region of the light chain of antibody number 1-20, which specifically recognizes the RBD.

DIQLTQSPSFLSASVGDRVTITCRASQGISSYLAWYQQKPGKAPKLLIYA
ASTLQSGVPSRFSGSGSGTEFTLTISSLQPEDFATYYCQQLNSYPCFGPG
TKVDIK

### Sequence of mAb 2-15

SEQ ID NO: 10 is the variable region of the heavy chain of antibody number 2-15.

QVQLVQSGAEVKKPGASVRVSCKASGYTFTGYYMHWVRQAPGQGLEWMGW
INPISDGTNYAQKFQGWVTMTRDTSISTVYMELSRLRSDDTAVYYCARGG
SRCSGGNCYGWAYDAFDIWGQGTMITVSS

SEQ ID NO: 11 is the variable region of the light chain of antibody number 2-15.

QSALTQPASVSGSPGQSITISCTGTSSDVGGYNFVSWYQQHPGKAPKLMI
YDVSKRPSGVSNRFSGSKSGNTASLTISGLQAEDEADCYCSSYTSSSTFV
FGTGTKVTVL

### Sequence of mAb 2-43

SEQ ID NO: 12 is the variable region of the heavy chain of antibody number 2-43, which recognizes an epitope other than the RBD or the NTD.

QVQLVQSGAEVKKPGASVKVSCKASGYTFTGYYMHWVRQAPGQGLEWMGW
INPNSGGTNYAQKFQGRVTMTRDTSITTAYMELRRLRSDDTAVYYCARGL
GVGCSGGNCYLDYYYMDVWGKGTTVTVSS

SEQ ID NO: 13 is the variable region of the light chain of antibody number 2-43, which recognizes an epitope other than the RBD or the NTD.

QSALTQPASVSGSPGQSITISCTGTSSDVGGYNYVSWYQQHPGKAPKLMI
YDVSKRPSGVSNRFSGSKSGNTASLTISGLQAEDEGDYYCSSYTSSSTWV
FGGGTKLTVL

### Sequence of mAb 1-57

SEQ ID NO: 14 is the variable region of the heavy chain of antibody number 1-57, which specifically recognizes the RBD.

EVHLVESGGGLVQPGGSLRLSCAASGFTFSDHYMDWVRQAPGKGLEWVGR
TRNKANSYTTEYAASVKGRFTISRDDAKNSLYLQMNSLKTEDTAVYYCAR
VHRWAYCINGVCFGAYSDYWGQGTLVTVSS

SEQ ID NO: 15 is the variable region of the light chain of antibody number 1-57, which specifically recognizes the RBD.

EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPSTFG
QGTKLEIK

### Sequence of mAb 2-30

SEQ ID NO: 16 is the variable region of the heavy chain of antibody number 2-30, which specifically recognizes the RBD.

QVQLVESGGGVVQPGRSLRLSCAASGFTFSSYTMHWVRQAPGKGLEWVAA
ISYDGNNKYYADSVKGRFTISRDNSKNTLYLQMNSLRAEDTAVYYCARSI
LYGGGMDVWGQGTTVTVSS

SEQ ID NO: 17 is the variable region of the light chain of antibody number 2-30, which specifically recognizes the RBD.

DIQLTQSPSSLSASVGDRVTITCRASQGISSYLAWYQQKPGQAPKLLIYA
ASTLQSGVPSRFSGSGSGTDFTLTISSLQPEDFATYYCQQLNSYPLTFGG
GTKVEIK

### Sequence of mAb 4-8

SEQ ID NO: 18 is the variable region of the heavy chain of antibody number 4-8.

QVQLVQSGAEVKKAGSSVKVSCKASGGTFSSHTITWVRQAPGQGLEWMGR
IIPILGIANYAQKFQGRVTITADKSTSTAYMELSSLRSEDTAVYYCASLQ
TVDTAIEKYYGMDVWGQGTTVTVSS

SEQ ID NO: 19 is the variable region of the light chain of antibody number 4-8

SYELTQPPSVSVSPGQTASITCSGDKLGDKYACWYQQKPGQSPVLVIYQD
NKRPSGIPERFSGSNSGNTATLTISGTQAMDEADYYCQAWDSSTAVFGGG
TKLTVL

### Sequence of mAb 5-24

SEQ ID NO: 20 is the variable region of the heavy chain of antibody number 5-24, which specifically recognizes the NTD.

QVQLVESGGGVVQPGRSLRLSCAASGLTFSSYVMHWVRQAPGKGLDWVGV
TWYDGSKKYYADSVKGRFTISRDNSKNTLYLQMNSLRAEDTAVYYCARDP
RDYYDEWSGYDYYYGLDVWGQGTTVTVSS

SEQ ID NO: 21 is the variable region of the light chain of antibody number 5-24, which specifically recognizes the NTD.

EIVLTQSPGILSLSPGERATLSCRASQSVSSSYLAWYQQKPGQAPRLLIY
GASSRATGIPDRFSGSGSGTDFTLTISRLEPEDFAVYYC
QQYGSSGALTFGGGTKVEIK

### Sequence of mAb 1-68

SEQ ID NO: 22 is the variable region of the heavy chain of antibody number 1-68, which specifically recognize the NTD.

QVQLVQSGAEVKKPGASVKVSCKVSGYTLIELSMHWVRQAPGKG
LEWMGGFDPEDAETIYAQKFQGRVTMTEDTSTDTAYMELSSLRS
EDTAVYYCATGWAVAGSSDVWYYYYGMDVWGQGTTVTVSS

SEQ ID NO: 23 is the variable region of the light chain of antibody number 1-68, which specifically recognizes the NTD.

QSALTQPPSVSGSPGQSVTISCTGTSSDVGSYNRVS
WYQQPPGTAPKLMIYEVSNRPSGVPDRFSGSKSGNT
ASLTISGLQAEDEADYYCSSYTSSSTYVFGTGTKVTVL

### Sequence of mAb 1-87

SEQ ID NO: 24 is the variable region of the heavy chain of antibody number 1-87, which specifically recognizes the NTD.

QVQLVQSGAEVKKPGASVKVSCKVSGYTLIELSMH
WVRQAPGKGLEWMGGFDPEDAETIYAQKFQGRVTM
TEDTSTDTAYMELSSLRSEDTAVYYCAT
GIAVIGPPPSTYYYYGMDVWGQGTTVTVSS

SEQ ID NO: 25 is the variable region of the light chain of antibody number 1-87, which specifically recognizes the NTD.

QSALTQPASVSGSPGQSITISCTGTSSDVGGYNYVS
WYQQHPGKAPKLMIYDVSKRPSGVSNRFSGSKSGNT
ASLTISGLQAEDEADYYCSSYTSSSTYVFGTGTKVTVL

### Sequence of mAb 2-51

SEQ ID NO: 26 is the variable region of the heavy chain of antibody number 2-51.

QVQLVQSGAEVKKPGASVKVSCKVSGYTLIELSMH
WVRQAPGKGLEWMGGFDPEDVETIYAQQFQG
RVTMTEDTSTDTAYMELSSLRSEDTAVYYCAT
GWAYKSTWYFGYWGQGTLVTVSS

SEQ ID NO: 27 is the variable region of the light chain of antibody number 2-51.

QSALTQPPSASGSPGQSVTISCTGTSSDVGGYNYVS
WYQQHPGKAPKLMIYEVSKRPSGVPDRFSGSKSGNT
ASLTVSGLQAEDEADYYCSSYAGSRMGFGGGTKLTVL

### Sequence of mAb 2-17

SEQ ID NO: 28 is the variable region of the heavy chain of antibody number 2-17, which recognizes an epitope other than the RBD or the NTD.

QVQLVQSGAEVKKPGSSVKVSCKASGGTFSSYAIS
WVRQAPGQGLEWMGGNIPIFGTANYAQKFQGRVTI
TADESTSTAYMELSSLRSEDTAVYYCAR
GVGYRGVIPLNWFDPWGQGTVVTVSS

SEQ ID NO: 29 is the variable region of the light chain of antibody number 2-17, which recognizes an epitope other than the RBD or the NTD.

EIVMTQSPATLSVSPGERATLSCRASQSVSSDLA
WYQHKPGQAPRLLIYGASTRATGIPVRFSGSGSG
TEFTLTISSLQSEDFAVYYCQQYNNWPPFT
FGGGTKVEIK

### Sequence of mAb 4-18

SEQ ID NO: 30 is the variable region of the heavy chain of antibody number 4-18, which specifically recognizes the NTD.

QVQLVESGGGVVQPGRSLRLSCAASGFTFSSYGMH
WVRQAPGKGLEWVAVISYDGSNKHYADSVKGRFTI
SRDNSKNTLYLQMNSLRAEDTAVYYCAK
DSGYNYGYSWFDPWGQGTLVTVSS

SEQ ID NO: 31 is the variable region of the light chain of antibody number 4-18, which specifically recognizes the NTD.

SYELTQPPSVSVSPGQTARITCSADALAKQYAY
WYQQKPGQAPVLVIYKDSERPSGIPERFSGSSS
GTTVTLTISGVQAEDEADYYCQSTDNSGTYPNWV
FGGGTKLTVL

### Sequence of mAb 4-19

SEQ ID NO: 32 is the variable region of the heavy chain of antibody number 4-19, which recognizes an epitope other than the RBD or the NTD.

QVQLQESGPGLVKPSETLSLTCTVSGGSISNYYWS
WIRQSPGKGLEWIGYIYYSGSTNYNPSLKSRVTIS
VDTSKNQFSLKLSSVTAADTAVYYCAR
SAKHWLAPPGDYYYYMDVWGKGTTVTVSS

SEQ ID NO: 33 is the variable region of the light chain of antibody number 4-19, which recognizes an epitope other than the RBD or the NTD.

DIQLTQSPSFLSASVDRVTITCRASQGISSYLA
WYQQKPGKAPKLLIYAASTLQSGVPSRFSGSGSG
TEFTLTISSLQPEDFATYYCQQLNSYLTFGGGTKVEIK

### Sequence of mAb 5-7

SEQ ID NO: 34 is the variable region of the heavy chain of antibody number 5-7, which specifically recognizes the NTD.

QVQLVQSGAEVKKPGASVKVSCKASGYTFTSYYMH
WVRQAPGQGLEWMGVINPSGGSTSYAEKFRGRVTM
TRDTSTSTVYMELSSLRSEDTAVYYCAR
DREPHSDSSGYWDSLKYYYYYALDVWGQGTTVTVSS

SEQ ID NO: 35 is the variable region of the light chain of antibody number 5-7, which specifically recognizes the NTD.

AIQLTQSPSSLSASVGDRVTITCRASQGISSYLA
WYQQKPGKAPELLIYAASTLQSGVPSRFSGSGSG
TDFTLTISSLQPEDFATYYCQQLNTYPFTFGPGTKVDIK

In some embodiments, the subject matter described herein reports the isolation, characterization and sequences of potent monoclonal antibodies (mAbs) against SARS-COV-2 and variants including the B. 1.351.2-7 variant. The mAbs can be isolated from blood samples from patients with COVID-19, and once the antibody sequences are obtained from these samples, the mAbs can be synthesized in vitro.

The SARS-COV-2 neutralizing mAbs described herein can be used for treatment of subjects infected with SARS-COV-2. In some embodiments, the mAbs described herein reduce SARS-COV-2 viral load in a subject in need thereof. In some embodiment, the mAbs described herein decrease disease severity in a subject in need thereof. In some embodiments, the mAbs described herein improve clinical outcome of COVID-19 in subjects in need thereof. In some embodiments, the mAbs described herein can be used as prophylaxis to prevent high risk subjects and individuals from becoming infected with SARS-COV-2.

Flow Through of Identification Process for the Potent COVID-19 mAbs Disclosed Herein
1. Plasma samples from COVID-19 patients were isolated and evaluated for the ability of potential antibodies contained within these plasma samples to neutralize SARS-CoV-2 and variants in vitro.
2. Peripheral blood mononuclear cells (PMBCs) were isolated from patients to isolate single B cells containing mAb-sequences that could be responsible for strong plasma antiviral activity. The antibody sequences were recovered from these single B cells by high throughput sequencing.
3. Genes from single B cells encoding for these mAb sequences were synthesized and cloned into expression vectors. mAbs were expressed in vitro and purified for subsequent analyses and characterization.
4. In vitro-produced and purified mAbs were then tested for their ability to neutralize an infection cause by SARS-COV-2 or a variant virus.

In some embodiments, the mAb described herein can be used for treatment and/or prevention of COVID-19. In some embodiment, the mAbs can be used for diagnosis of COVID-19 subject exposure. In some embodiment, the mAbs described herein can be utilized in laboratory research and development activities.

Immunoglobulins or antibodies are Y-shaped proteins consisting of two identical light chains (LCs) and two identical heavy chains (HCs) of amino acid sequences. The light and heavy chains associates with each other to form the intact immunoglobulin heterodimer. In some embodiments, the HC and LC of the heterodimer are linked through disulfide bonds. In some embodiments, the two HCs of the heterotetramer are linked by disulfide bridges. Human LCs can be one of two functionally similar classes, K or A. Both LC classes have two domains, a constant domain (CL) and a variable domain (VL). Human antibody HCs can be one of five isotypes, IgA, IgD, IgE, IgG, and IgM. IgAs, IgDs, and IgGs have three constant (C) and one variable (V) domains. IgEs and IgMs have one variable and four constant domains. The IgA and IgM isotopes have an additional J-chain, which allows the formation of dimers and pentamers, respectively.

An antibody molecule has three functional components, two Fragment antigen binding domains (Fabs) and the fragment crystallizable (Fc), with the two Fabs linked to the Fc by a hinge region that allows the Fabs a large degree of flexibility relative to the Fc. In monoclonal antibodies, each of the Fabs have identical antigen-binding sites for binding to a specific target antigen. The Fv region of the Fab is composed of a pair of variable domains (variable heavy (VH) and variable light (VL)) contributed by the heavy chain (HC) and the light chain (LC). In some embodiments, the domains of heavy and light chains are approximately 110 amino acid residues in length. The Fv domains of the immunoglobulin structure, which interact with the target antigen, are at the N-termini of the HCs and LCs. The Fab regions of an immunoglobulin molecule are formed by the pairing of VL and constant light (CL) of the LCs with VH and the first constant heavy region (CH1) of the HCs. The pairing of VL and VH, form the antigen-binding site.

The polypeptide region connecting the Fab and the Fc regions called the hinge region. The hinge region functionally allows the Fabs a large degree of conformational flexibility relative to the Fc. The antibody hinge can be divided into three regions, the upper hinge, core hinge, and lower hinge, each with a different functional role. The upper hinge allows the movement and rotation of the Fabs. The central core hinge contains a variable number of cysteine residues depending on the IgG subtype that forms disulfide bonds, stabilizing the association of the two heavy chains. The lower hinge that allows movement of the Fc relative to the Fabs.

### Example 1—Neutralizing Antibodies Against SARS-COV-2 and its Variants

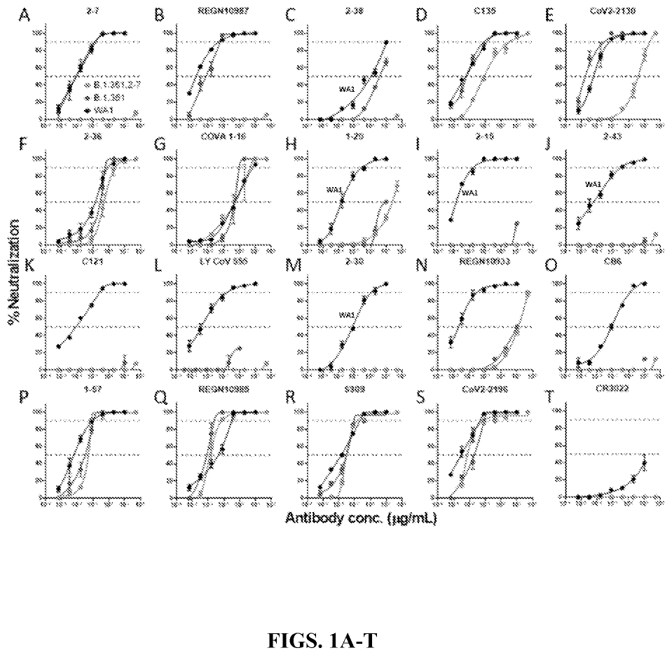

FIGS. 1A-T show a neutralization of 2-7 resistant B.1.351 (B.1.351.2-7) virus strain with monoclonal antibodies to the receptor binding domain (RBD) of the B. 1.351.2-7 spike protein. FIG. 1A shows neutralization for mAb 2-7. FIG. 1B shows neutralization for mAb REGN10987. FIG. 1C shows neutralization for mAb 2-38. FIG. 1D shows neutralization for mAb C135. FIG. 1E shows neutralization for mAb CoV2-2130. FIG. 1F shows neutralization for mAb 2-36. FIG. 1G shows neutralization for mAb COVA 1-16. FIG. 1H shows neutralization for mAb 1-20. FIG. 1I shows neutralization for mAb 2-15. FIG. 1J shows neutralization for mAb 2-43. FIG. 1K shows neutralization for mAb C121. FIG. 1L shows neutralization for mAb LY CoV 555. FIG. 1M shows neutralization for mAb 2-30. FIG. 1N shows neutralization for mAb REGN10985. FIG. 1O shows neutralization for mAb CB6. FIG. 1P shows neutralization for mAb 1-57. FIG. 1Q shows neutralization for mAb REGN10985. FIG. 1R shows neutralization for mAb S309. FIG. 1S shows neutralization for mAb CoV2-2196. FIG. 1T shows neutralization for mAb CR3022. Also shown are the neutralization curves for the SARS-COV-2 wild-type virus and the B.1.351 virus variant.

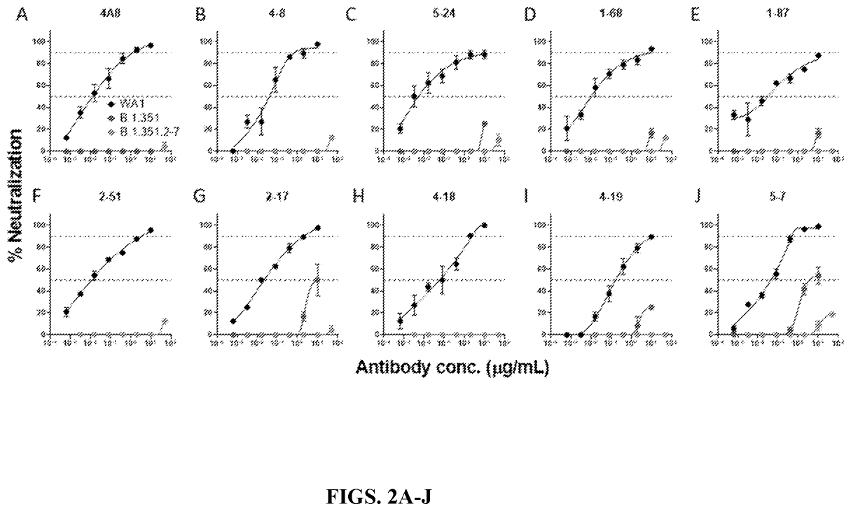

FIGS. 2A-J shows neutralization of 2-7 resistant B.1.351 (B.1.351.2-7) virus strain with monoclonal antibodies (mAb) to the N-terminal domain (NTD) of the B.1.351.2-7 spike protein. FIG. 1A shows neutralization for mAb 4A8. FIG. 1B shows neutralization for mAb 4-8. FIG. 1C shows neutralization for mAb 5-24. FIG. 1D shows neutralization for mAb 1-68. FIG. 1E shows neutralization for mAb 1-87. FIG. 1F shows neutralization for mAb 2-51. FIG. 1G shows neutralization for mAb 2-17. FIG. 1H shows neutralization for mAb 4-18. FIG. 1I shows neutralization for mAb 4-19. FIG. 1J shows neutralization for mAb 5-7. Also shown are the neutralization curves for the SARS-COV-2 wild-type virus and the B. 1.351 virus variant.

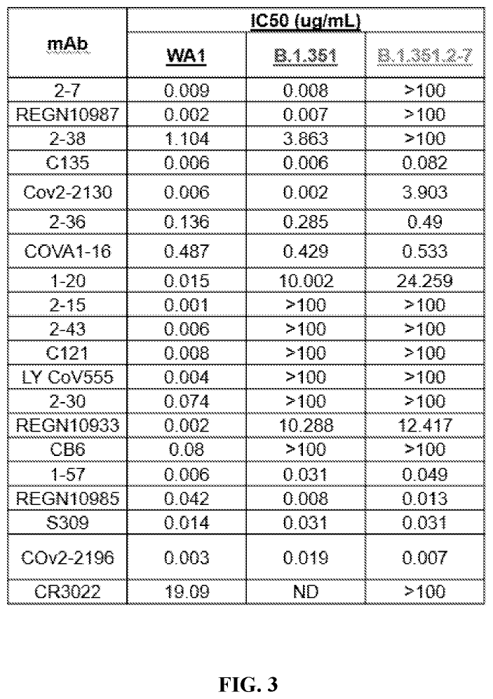

FIG. 3 shows a range of potency (IC₅₀ μg/ml) of RBD mAbs to the WA1, B.1.351, and the B.1.351.2-7 virus strains.

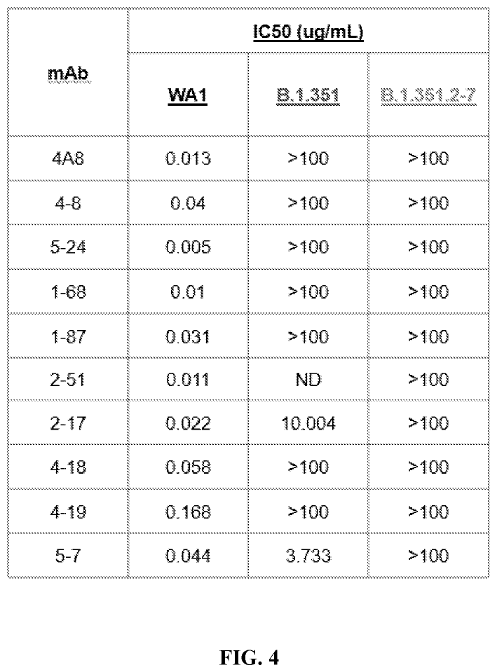

FIG. 4 shows neutralization (IC₅₀ μg/ml) of NTD mAbs to the WA1, B. 1.351, and the B.1.351.2-7 virus strains.

FIG. 5 shows a schematic representation of the SARS-COV-2 B.1.351.2-7 variant mutations. FIG. 5 also shows the key differences in spike protein sequence from strain WA1 to strain B1.351 and to strain B.1.351.2-7. In the NTD of the spike protein sequence for the B.1.351.2-7 stain, amino acids L241, L242, and A243 are deleted while R66 has replaced H66. In the RBD of the spike protein sequence for the B.1.351.2-7 strain, amino acid G445 has replaced V445 as compared to the B.1.351 strain. In the S1/S2 domains of the spike protein sequence for the B.1.351.2-7 stain, amino acids 677-681 are deleted while G614 has replaced D614 as compared to the WA1 strain.

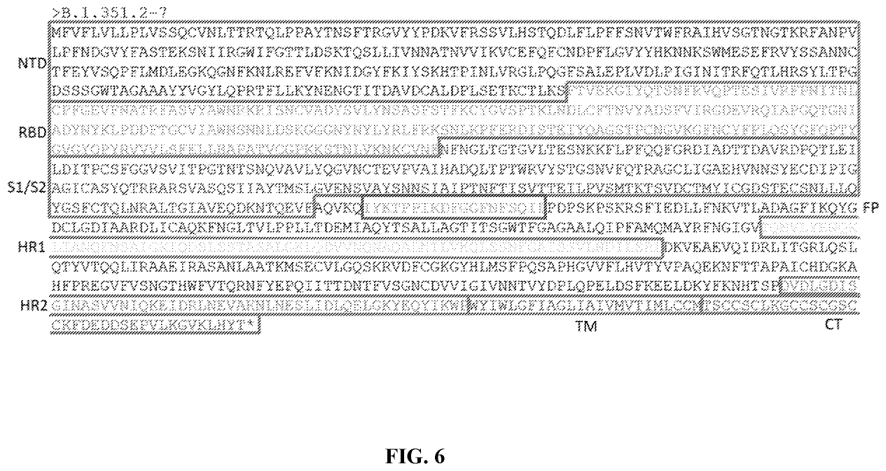

FIG. 6 shows the sequence of the B.1.351.2-7 spike protein. Yellow highlight indicates the NTD. Green font indicates the RBD. Dark blue font indicates the S1/S2 domains. Light blue font is the FP domain. Orange font indicates the HR1 domain. Brown font indicates the HR2 domain. Purple font is the TM domain. Pink font is the CT domain. A label for each domain can be found next to its corresponding amino acid sequence. The sequence for the FP domain is in the center of the corresponding line.

### Example 2—DNA-Based Vaccine Against B. 1.351.2-7

In some embodiments, the subject matter disclosed herein relates to a vaccine that induces an immune response against severe acute respiratory syndrome coronavirus (SARS-CoV-2). In some embodiments, the vaccine is administered as prophylaxis against a SARS-CoV-2 virus, which can invade susceptible host organisms and cause disease therein. In some embodiments, the vaccine is administered to treat diseases caused by the infection. In some embodiments, the vaccine contains a nucleic acid sequence encoding at least a portion of a SARS-COV-2 spike protein. In some embodiments, the SARS-COV-2 spike protein is a B.1.351.2-7 variant spike protein. In some embodiments, the vaccine contains a nucleic acid sequence encoding at least a portion of the amino acid sequence defined by SEQ ID NO: 1. In some embodiments, the vaccine contains a nucleic acid sequence encoding an amino acid sequence which is 50%, 55%, 60%, 65%, 70%, 75%, 80%, 85%, 90%, or 95% identical to an amino acid sequence defined by SEQ ID NO: 1. In some embodiments, the vaccine contains a nucleic acid sequence encoding the amino acid sequence defined by SEQ ID NO: 1.

In some embodiments, the host animals in which the vaccine disclosed herein may induce the immune response against SARS-COV-2 include, but are not limited to, mammals or birds, for example, humans, dogs, cats, pigs, horses, chickens, ducks, turkeys, ferrets, and the like.

The vaccine described herein may additionally contain, but is not limited to, at least one component selected from the group consisting of solvent, adjuvant and/or excipient. The solvent may include physiological saline or distilled water. The adjuvant may include Freund's incomplete or complete adjuvants, aluminum hydroxide gels, and vegetable and mineral oils. Excipients may include aluminum phosphate, aluminum hydroxide, or aluminum potassium sulfate. In some embodiments, the vaccine disclosed herein contain at least one substance used in vaccine preparation, which is well known to those skilled in the art.

In some embodiments, the composition of the vaccine described herein is adjusted for administrations depending on the subject's body weight, age, severity of symptoms, or other physical characteristics. In some embodiments, the vaccine described herein may be prepared in oral or parenteral formulations. In some embodiments, the vaccine may be administered via intradermal, intramuscular, intraperitoneal, intranasally or epidural route. In some embodiments, the vaccine is administered by any suitable route known in the art.

In some embodiments, the subject matter described herein relates to a method of preventing or treating SARS-COV-2 or variant SARS-COV-2 infections by administering the vaccine described herein to one or more subjects. Such infections can lead to COVID-19 symptoms or disease in subjects. The term “subject” as used herein refers to any animal including a human that has already been infected or may be infected with a SARS-COV-2 virus or a variant virus. In some embodiments, the SARS-COV-2 variants contain at least one amino acid mutation in the sequence encoding the spike protein is SARS-COV-2 compared to wild-type SARS-COV-1. In some embodiments, the vaccine described herein may be administered alone or in combination with any COVID-19 treatment agent. In some embodiments, the vaccine can be administered with any agents used in the art to treat infections.

As used herein, the term “prevention” refers to any action that inhibits infection with SARS-COV-2 or a variant strain or delays development or decreases severity of disease caused by the infection via administration of the vaccine described herein.

As used herein, the term “treatment” refers to any action in which symptoms caused by SARS-COV-2 or variant strain infection are reduced or beneficially altered via administration of the vaccine described herein.

In some embodiments, the vaccine described herein is administered as an individual therapeutic agent or administered in combination with other therapeutic agents. In some embodiments, the vaccine may be administered sequentially or simultaneously with a conventional therapeutic agent. In some embodiments, the vaccine may be administered in a single or multiple manner.

In one embodiment, the subject matter disclosed herein relates to the generation and validation of a vaccine against the B.1.351.2-7 variant of the SARS-COV-2 virus or other emerging SARS-COV-2 variants. In one embodiment, the vaccine is based on the DNA nucleotide sequence encoding at least a portion of the B.1.351.2-7 spike protein (SEQ ID NO: 1).

In one embodiment, the DNA-based vaccine against B.1.351.2-7 introduces a genetically engineered plasmid containing the DNA sequence encoding at least a portion of the B.1.351.2-7 spike protein (SEQ ID NO: 1) into a subject in need thereof. In some embodiments, the vaccine contains a DNA nucleic acid sequence encoding at least a portion of a SARS-COV-2 spike protein. In some embodiments, the SARS-COV-2 spike protein is a B.1.351.2-7 variant spike protein. In some embodiments, the vaccine contains a nucleic acid sequence encoding at least a portion of the amino acid sequence defined by SEQ ID NO: 1. In some embodiments, the vaccine contains a nucleic acid sequence encoding an amino acid sequence which is 50%, 55%, 60%, 65%, 70%, 75%, 80%, 85%, 90%, or 95% identical to an amino acid sequence defined by SEQ ID NO: 1. In some embodiments, the vaccine contains a nucleic acid sequence encoding the amino acid sequence defined by SEQ ID NO: 1. In some embodiments, the introduction is via injection. In some embodiments, the introduction is via a nasal spray. In some embodiments, the introduction is via any suitable method know in the art. In some embodiments, the vaccination is to prevent the spread of the B.1.351.2-7 variant. In some embodiments, the vaccination is to treat a subject that has tested positive for B.1.351.2-7. In some embodiments, the subject has been exposed to B.1.351.2-7. In some embodiments, the subject exhibits COVID-19 symptoms. In some embodiments, the subject has been diagnosed with COVID-19.

Following the inoculation of the subject with the DNA-based vaccine described herein, the subject's cells directly produce the B.1.351.2-7 spike protein or at least the portion encoded by the DNA. This causes a protective immunological response to be generated in the subject's systems. When the vaccinated subject is infected with the B.1.351.2-7 virus, severe disease can be avoided. In some embodiments, the vaccinated subject infected with B.1.351.2-7 shows no symptoms of disease or very mild symptoms. In some embodiments, the vaccinated subject does not transmit the virus to other subjects. In some embodiments, the DNA vaccine can induce a wide range of immune response types.

In some embodiments, the vaccination regimen includes one or more events of introducing the B.1.351.2-7 DNA into the subject. In some embodiments, the introduction events are spread over time. In some embodiments, the initial vaccination is followed by at least one booster shot within a month of the initial vaccination. In some embodiments, the initial vaccine and the booster shots are based on the same B.1.351.2-7 DNA spike protein sequence encoding at least a portion of the B.1.351.2-7 spike protein. In some embodiments, the initial vaccine and the booster shots are based on different B.1.351.2-7 DNA spike protein sequences, each encoding at least a portion of the B.1.351.2-7 spike protein.

### Example 3—RNA Vaccine Against B.1.351.2-7

In some embodiments, the vaccine described herein is based on the mRNA nucleotide sequence encoding at least a portion of the B.1.351.2-7 spike protein (SEQ ID NO: 1). In some embodiments, the vaccine contains a mRNA nucleic acid encoding at least a portion of a SARS-COV-2 spike protein. In some embodiments, the SARS-COV-2 spike protein is a B.1.351.2-7 variant spike protein. In some embodiments, the vaccine contains a mRNA nucleic acid encoding at least a portion of the amino acid defined by SEQ ID NO: 1. In some embodiments, the vaccine contains a mRNA nucleic acid encoding an amino acid sequence which is 50%, 55%, 60%, 65%, 70%, 75%, 80%, 85%, 90%, or 95% identical to an amino acid sequence defined by SEQ ID NO: 1. In some embodiments, the vaccine contains a nucleic acid sequence encoding the amino acid sequence defined by SEQ ID NO: 1.

The mRNA strand introduced into the subject enters the body's cells and the cells use the genetic information encoded by the mRNA to produce the B.1.351.2-7 spike protein or the encoded portion thereof. The subject's immune system then learns to recognize the protein. RNA vaccines can be delivered by a variety of methods including but not limited to needle-syringe injections or needle-free into the skin; via injection into the blood, muscle, lymph node or directly into organs; or via a nasal spray.

In some embodiments, the vaccination regimen includes one or more events of delivering the B.1.351.2-7 RNA, the events being spread over time. In some embodiments, the initial vaccination is followed by at least one booster shot within a month of the initial vaccination. In some embodiments, the initial vaccine and the booster shots are based on the same B.1.351.2-7 RNA sequence encoding at least a portion of the B.1.351.2-7 spike protein. In some embodiments, the initial vaccine and the booster shots are based on different B.1.351.2-7 RNA sequences, each encoding at least a portion of the B.1.351.2-7 spike protein.

What is claimed is:

1. An engineered antibody or a functional fragment thereof, wherein the antibody or fragment thereof selectively binds at least a portion of a SARS-COV-2 spike protein.

2. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 4 and a second amino acid sequence having 50%, 55%, 60%, 65%, 70%, 75%, 80%, 85%, 90%, or 95% identity with SEQ ID NO: 5.

3. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 6 and a second amino acid sequence having 50%, 55%, 60%, 65%, 70%, 75%, 80%, 85%, 90%, or 95% identity with SEQ ID NO: 7.

4. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 8 and a second amino acid sequence having 50%, 55%, 60%, 65%, 70%, 75%, 80%, 85%, 90%, or 95% identity with SEQ ID NO: 9.

5. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 12 and a second amino acid sequence having 50%, 55%, 60%, 65%, 70%, 75%, 80%, 85%, 90%, or 95% identity with SEQ ID NO: 13.

6. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 16 and a second amino acid sequence having 50%, 55%, 60%, 65%, 70%, 75%, 80%, 85%, 90%, or 95% identity with SEQ ID NO: 17.

7. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 18 and a second amino acid sequence having 50%, 55%, 60%, 65%, 70%, 75%, 80%, 85%, 90%, or 95% identity with SEQ ID NO: 19.

8. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 20 and a second amino acid sequence having 50%, 55%, 60%, 65%, 70%, 75%, 80%, 85%, 90%, or 95% identity with SEQ ID NO: 21.

9. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 22 and a second amino acid sequence having 50%, 55%, 60%, 65%, 70%, 75%, 80%, 85%, 90%, or 95% identity with SEQ ID NO: 23.

10. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 24 and a second amino acid sequence having 50%, 55%, 60%, 65%, 70%, 75%, 80%, 85%, 90%, or 95% identity with SEQ ID NO: 25.

11. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 26 and a second amino acid sequence having 50%, 55%, 60%, 65%, 70%, 75%, 80%, 85%, 90%, or 95% identity with SEQ ID NO: 27.

12. The antibody or fragment of claim 1, wherein the antibody or fragment comprises a first amino acid sequence having 50%, 55%, 60%, 65%, 70%, 75%, 80%, 85%, 90%, or 95% identity with SEQ ID NO: 32 and a second amino acid sequence having 50%, 55%, 60%, 65%, 70%, 75%, 80%, 85%, 90%, or 95% identity with SEQ ID NO: 33.

13. The antibody or fragment of claim 1, wherein the antibody or fragment comprises a first amino acid sequence comprising SEQ ID NO: 4 and a second amino acid sequence comprising SEQ ID NO: 5.

14. The antibody or fragment of claim 1, wherein the antibody or fragment comprises a first amino acid sequence comprising SEQ ID NO: 6 and a second amino acid sequence comprising SEQ ID NO: 7.

15. The antibody or fragment of claim 1, wherein the antibody or fragment comprises a first amino acid sequence comprising SEQ ID NO: 8 and a second amino acid sequence comprising SEQ ID NO: 9.

16. The antibody or fragment of claim 1, wherein the antibody or fragment comprises a first amino acid sequence comprising SEQ ID NO: 12 and a second amino acid sequence comprising SEQ ID NO: 13.

17. The antibody or fragment of claim 1, wherein the antibody or fragment comprises a first amino acid sequence comprising SEQ ID NO: 16 and a second amino acid sequence comprising SEQ ID NO: 17.

18. The antibody or fragment of claim 1, wherein the antibody or fragment comprises a first amino acid sequence comprising SEQ ID NO: 18 and a second amino acid sequence comprising SEQ ID NO: 19.

19. The antibody or fragment of claim 1, wherein the antibody or fragment comprises a first amino acid sequence comprising SEQ ID NO: 20 and a second amino acid sequence comprising SEQ ID NO: 21.

20. The antibody or fragment of claim 1, wherein the antibody or fragment comprises a first amino acid sequence comprising SEQ ID NO: 22 and a second amino acid sequence comprising SEQ ID NO: 23.

21. The antibody or fragment of claim 1, wherein the antibody or fragment comprises a first amino acid sequence comprising SEQ ID NO: 24 and a second amino acid sequence comprising SEQ ID NO: 25.

22. The antibody or fragment of claim 1, wherein the antibody or fragment comprises a first amino acid sequence comprising SEQ ID NO: 26 and a second amino acid sequence comprising SEQ ID NO: 27.

23. The antibody or fragment of claim 1, wherein the antibody or fragment comprises a first amino acid sequence comprising SEQ ID NO: 32 and a second amino acid sequence comprising SEQ ID NO: 33.

24. The antibody or fragment of claim 1, wherein the antibody or fragment comprises the complementarity-determining regions (CDRs) of SEQ ID NO: 4 and the complementarity-determining regions (CDRs) of SEQ ID NO: 5.

25. The antibody or fragment of claim 1, wherein the antibody or fragment comprises the complementarity-determining regions (CDRs) of SEQ ID NO: 6 and the complementarity-determining regions (CDRs) of SEQ ID NO: 7.

26. The antibody or fragment of claim 1, wherein the antibody or fragment comprises the complementarity-determining regions (CDRs) of SEQ ID NO: 8 and the complementarity-determining regions (CDRs) of SEQ ID NO: 9.

27. The antibody or fragment of claim 1, wherein the antibody or fragment comprises the complementarity-determining regions (CDRs) of SEQ ID NO: 12 and the complementarity-determining regions (CDRs) of SEQ ID NO: 13.

28. The antibody or fragment of claim 1, wherein the antibody or fragment comprises the complementarity-determining regions (CDRs) of SEQ ID NO: 16 and the complementarity-determining regions (CDRs) of SEQ ID NO: 17.

29. The antibody or fragment of claim 1, wherein the antibody or fragment comprises the complementarity-determining regions (CDRs) of SEQ ID NO: 18 and the complementarity-determining regions (CDRs) of SEQ ID NO: 19.

30. The antibody or fragment of claim 1, wherein the antibody or fragment comprises the complementarity-determining regions (CDRs) of SEQ ID NO: 20 and the complementarity-determining regions (CDRs) of SEQ ID NO: 21.

31. The antibody or fragment of claim 1, wherein the antibody or fragment comprises the complementarity-determining regions (CDRs) of SEQ ID NO: 22 and the complementarity-determining regions (CDRs) of SEQ ID NO: 23.

32. The antibody or fragment of claim 1, wherein the antibody or fragment comprises the complementarity-determining regions (CDRs) of SEQ ID NO: 24 and the complementarity-determining regions (CDRs) of SEQ ID NO: 25.

33. The antibody or fragment of claim 1, wherein the antibody or fragment comprises the complementarity-determining regions (CDRs) of SEQ ID NO: 26 and the complementarity-determining regions (CDRs) of SEQ ID NO: 27.

34. The antibody or fragment of claim 1, wherein the antibody or fragment comprises the complementarity-determining regions (CDRs) of SEQ ID NO: 32 and the complementarity-determining regions (CDRs) of SEQ ID NO: 33.

35. The antibody or fragment of any one of claims 1-34, wherein the SARS-COV-2 spike protein is a wild-type SARS-COV-2 WA1 spike protein.

36. The antibody or fragment of any one of claims 1-34, wherein the SARS-COV-2 spike protein is a SARS-COV-2 B.1.351 variant spike protein.

37. The antibody or fragment of any one of claims 1-34, wherein the SARS-COV-2 spike protein is a SARS-COV-2 B.1.351.2-7 variant spike protein.

38. The antibody or fragment of any one of claims 1-34, wherein the SARS-COV-2 spike protein is a SARS-COV-2 B.1.351.2-7 variant spike protein comprising SEQ ID NO: 1.

39. The antibody or fragment of any one of claims 1-34, wherein the antibody neutralizes a SARS-COV-2 virus.

40. The antibody or fragment of any one of claims 1-34, wherein the antibody neutralizes a SARS-COV-2 B.1.351 variant virus.

41. The antibody or fragment of any one of claims 1-34, wherein the antibody a SARS-CoV-2 B.1.351.2-7 variant virus.

42. A method for treating a subject having a viral infection or preventing a subject from developing a viral infection, the method comprising administering to the subject a therapeutically effective amount of a composition comprising the engineered monoclonal antibody or fragment of any one of claims 1-34, wherein the antibody or fragment selectively binds at least a portion of a SARS-COV-2 spike protein.

43. The method of claim 42, wherein the SARS-COV-2 spike protein is a wild-type SARS-COV-2 WA1 spike protein.

44. The method of claim 42, wherein the SARS-COV-2 spike protein is a SARS-COV-2 B.1.351 variant spike protein.

45. The method of claim 42, wherein the SARS-COV-2 spike protein is a SARS-COV-2 B.1.351.2-7 variant spike protein.

46. The method of claim 42, wherein the SARS-COV-2 spike protein is a SARS-COV-2 B.1.351.2-7 variant spike protein comprising SEQ ID NO: 1.

47. The method of claim 42, wherein the antibody neutralizes a SARS-COV-2 virus.

48. The method of claim 42, wherein the antibody neutralizes a SARS-COV-2 B.1.351 variant virus.

49. The method of claim 42, wherein the antibody a SARS-COV-2 B.1.351.2-7 variant virus.

50. A purified protein, wherein the protein comprises at least a portion of a SARS-COV-2 surface protein.

51. The protein of claim 50, wherein the at least a portion of a SARS-COV-2 surface protein is a B.1.351.2-7 variant surface protein.

52. The protein of claim 51, wherein the B.1.351.2-7 variant surface protein is a spike protein (S).

53. The protein of claim 52, wherein the spike protein comprises an amino acid sequence having 50%, 55%, 60%, 65%, 70%, 75%, 80%, 85%, 90%, or 95% identity with SEQ ID NO: 1.

54. The protein of claim 52, wherein the spike protein comprises SEQ ID NO: 1.

55. A synthetic nucleic acid sequence comprising a nucleotide sequence encoding a SARS-COV-2 B.1.351.2-7 variant spike protein.

56. The nucleic acid of claim 55, wherein the nucleic acid sequence is a complementary deoxyribonucleic acid (cDNA) encoding for a SARS-COV-2 B.1.351.2-7 variant spike protein comprising SEQ ID NO: 1.

57. The nucleic acid of claim 55, wherein the nucleic acid sequence is a messenger ribonucleic acid (mRNA) encoding for a SARS-COV-2 B.1.351.2-7 variant spike protein comprising SEQ ID NO: 1.

58. The nucleic acid of claim 55, wherein the nucleic acid sequence encodes a B.1.351.2-7 variant spike protein comprising an amino acid sequence having 50%, 55%, 60%, 65%, 70%, 75%, 80%, 85%, 90%, or 95% identity with SEQ ID NO: 1.

59. The nucleic acid of claim 55, wherein the nucleic acid sequence encodes a B.1.351.2-7 variant spike protein comprising SEQ ID NO: 1.

60. A pharmaceutical composition comprising a purified spike protein encoded by an amino acid sequence defined by SEQ ID NO: 1 or a portion thereof and a carrier, wherein the composition induces an immune response in a subject in need thereof.

61. A pharmaceutical composition comprising:
(i) a synthetic nucleic acid sequence comprising a nucleotide sequence encoding a SARS-COV-2 B.1.351.2-7 variant spike protein or a portion thereof; and
(ii) a carrier.

62. The composition of claim 61, wherein the nucleic acid sequence is a complementary deoxyribonucleic acid (cDNA) encoding for the SARS-COV-2 B.1.351.2-7 variant spike protein comprising SEQ ID NO: 1.

63. The composition of claim 61, wherein the nucleic acid sequence is a messenger ribonucleic acid (mRNA) encoding for the SARS-COV-2 B.1.351.2-7 variant spike protein comprising SEQ ID NO: 1.

64. The composition of claim 61, wherein the nucleic acid sequence encodes a B.1.351.2-7 variant spike protein comprising an amino acid sequence having 50%, 55%, 60%, 65%, 70%, 75%, 80%, 85%, 90%, or 95% identity with SEQ ID NO: 1.

65. The composition of claim 61, wherein the nucleic acid sequence encodes a B.1.351.2-7 variant spike protein comprising SEQ ID NO: 1.

66. The composition of any one of claims 61-65 further comprising an adjuvant.

67. A method of inducing an immune response in a subject in need thereof, the method comprising administering to the subject the composition of any one of claims 61-62.

68. The method of claim 67, wherein the subject is at risk of contracting a SARS-COV-2 virus.

69. The method of claim 68, wherein the SARS-COV-2 virus is a WA1 strain.

70. The method of claim 68, wherein the SARS-COV-2 virus is a B. 1.351 strain.

71. The method of claim 68, wherein the SARS-COV-2 virus is a B.1.351.2-7 strain.

72. The method of claim 67, wherein the method comprises a prime-boost regimen of administering the composition.

73. The method of claim 69, wherein the prime and the boost comprise the same composition.

74. The method of claim 69, wherein the prime and the boost comprise a different composition.
